(19) United States
(12) Patent Application Publication
(10) Pub. No.: US 2026/0216061 A1
(43) Pub. Date: Jul. 30, 2026

(54) RAPIDLY DISSOLVING VAGINAL FILM WITH CONTRACEPTIVE ACTION AND SEXUALLY TRANSMITTED INFECTION PREVENTION

(71) Applicant: EVOFEM, INC., San Diego, CA (US)

(72) Inventors: Sanjay GARG, San Diego, CA (US); Afinjuomo Olumide FRANKLIN, San Diego, CA (US); Yunmei SONG, San Diego, CA (US)

(21) Appl. No.: 18/705,917

(22) PCT Filed: Oct. 26, 2022
(86) PCT No.: PCT/US2022/078741

§ 371 Date: Apr. 29, 2024

Related U.S. Application Data
(60) Provisional application No. 63/273,013, filed on Oct. 28, 2021.

(51) Int. Cl. A61K 9/00; A61K 9/70; A61K 47/02; A61K 47/10; A61K 47/12; A61K 47/32; A61K 47/38

(52) U.S. Cl. CPC A61K 9/0034; A61K 9/7007; A61K 47/02; A61K 47/10; A61K 47/12; A61K 47/32; A61K 47/38

(57) ABSTRACT

Provided herein are thin film compositions and methods of making thin films comprising lactic acid (e.g. L-lactic acid), a water-soluble polymer, and a plasticizer. The thin films may be used for contraception and/or in preventing transmission of sexually transmitted infections.

### CROSS-REFERENCE TO RELATED APPLICATIONS

This application is a U.S. National Stage Entry of PCT/US2022/078741, filed on Oct. 26, 2022, which claims the benefit of U.S. Provisional Patent Application No. 63/273,013, filed Oct. 28, 2021, which is incorporated herein by reference in its entirety.

### BACKGROUND

The increasing incidence of sexually transmitted infections (STIs) globally requires an innovative drug delivery system to reduce the spread of HIV and sexually transmitted diseases. Further, there is a need for female-controlled, non-hormonal contraceptives. Many women cannot, or prefer not to, use hormonal birth control. There is a clear need to develop an effective woman-controlled method to address these issues.

The vaginal route can be used for mucosal drug delivery for both systemic and local applications. The use of vaginal routes allows the delivery of microbicides and other agents such as antiviral, antibacterial, spermicidal agents, prostaglandins, and steroids (Machado et al. J Pharm Sciences 102 (7): 2069-2081 (2013)). The advantage of using the vagina for drug delivery and contraception includes its large surface area, rich blood supply, and high permeability to many drugs. These characteristic make the vagina a potential site for the delivery of drugs and other active agents. Furthermore, the delivery of drugs to the vagina also allows avoidance of the first-pass metabolism as well as reduced adverse effects. Currently, the delivery of drugs to the vagina has been accomplished using gels, films, tablets, creams, suppositories, foam and vaginal rings (Furst et al. Eur J Pharm Biopharm 95:128-135 (2015); Ensign et al. J Control Rel 190:500-514 (2014)). The short residence time at the site of application for some of the vaginal dosage forms like gels, cream, and foam can be attributed partly to the self-cleaning ability of the vagina environment (Baloglu et al. J Pharm & Amp Pharm Sciences 12 (3): 312-336 (2009); Andrews et al. Biomacromolecules 10 (9): 2427-2435 (2009)) and results in poor therapeutic effect.

### SUMMARY

The use of vaginal film with mucoadhesive properties can address some of the current challenges with the traditional/conventional dosage forms. Furthermore, the design of the vaginal film allows for easy storage, stability, and convenience. The use of film will reduce other challenges such as poor compliance, leakages, and messiness. By utilizing mucoadhesive and film-forming polymers with an excellent binding capacity to the vaginal mucosa, the residence time of API or active agent can be increased.

The instant technology generally relates to compositions and methods for providing contraception. The instant technology also generally relates to compositions and methods for preventing the transmission of sexually transmitted infections.

In one aspect, the present disclosure provides a thin film comprising L-lactic acid, a water-soluble polymer, and a plasticizer.

In some embodiments, the composition further comprises an additional buffering agent. In some embodiments, the additional buffering agent is an organic acid. In some embodiments, the additional buffering agent is selected from citric acid, tartaric acid, benzoic acid, alginic acid, sodium monocitrate, sodium hydroxide and any combination thereof.

In some embodiments, the thin film composition further comprises a preservative. In some embodiments, the preservative is benzoic acid and/or sodium benzoate.

In some embodiments, the water-soluble polymer is selected from hydroxypropyl methylcellulose (HPMC), polyvinyl alcohol (PVA), xanthan gum, sodium alginate, carbopol, hydroxyethylcellulose, and any combination thereof. In some embodiments, the water-soluble polymer comprises HPMC. In some embodiments, the water-soluble polymer comprises PVA. In some embodiments, the water-soluble polymer comprises PVA and HPMC. In some embodiments, the water-soluble polymer comprises xanthan gum, PVA and HPMC. In some embodiments, the PVA has an average molecular weight between about 30,000 and about 186,000.

In some embodiments, the plasticizer is selected from glycerol, polyethylene glycol, propylene glycol, sorbitol, and any combination thereof. In some embodiments, the plasticizer is glycerol. In some embodiments, the glycerol is present at a concentration between about 0.2% (v/v) and about 4% (v/v).

In some embodiments, the L-lactic acid is present at a concentration between about 5 mg/mL and about 20 mg/mL. In some embodiments, the L-lactic acid is present at a concentration between about 8 mg/mL and about 15 mg/mL.

In some embodiments, the water-soluble polymer is present at a concentration between about 1 mg/mL and about 150 mg/mL.

In some embodiments, the thin film composition does not comprise alginic acid.

In another aspect, the present disclosure provides a thin film comprising PVA, HPMC, xanthan gum, citric acid, tartaric acid, benzoic acid, glycerol, L-lactic acid, and sodium hydroxide.

In some embodiments, the PVA has an average molecular weight between about 30,000 and about 186,000. In some embodiments, the thin film comprises about 40 mg/mL to about 80 mg/mL PVA having an average molecular weight of about 30,000 to about 70,000; about 0 mg/mL to about 10 mg/mL PVA having an average molecular weight of about 85,000 to about 124,000; and about 0 mg/mL and about 8 mg/mL PVA having an average molecular weight of about 146,000 to about 186,000. In some embodiments, the thin comprises about 40 mg/mL to about 80 mg/mL PVA having an average molecular weight of about 30,000 to about 70,000 and about 2 mg/mL to about 10 mg/mL PVA having an average molecular weight of about 85,000 to about 124,000.

In some embodiments, the thin film comprises about 40 mg/mL to about 80 mg/mL PVA having an average molecular weight of about 30,000 to about 70,000 and about 1 mg/mL to about 10 mg/mL PVA having an average molecular weight of about 146,000 to about 186,000.

In some embodiments, the thin film comprises about 1 mg/mL to about 10 mg/mL HPMC. In some embodiments, the HPMC is HPMC E50. In some embodiments, the HPMC is HPMC K15.

In some embodiments, the thin film may comprise between about 0.1 mg/mL and about 1.5 mg/mL xanthan gum. In some embodiments, the thin film may comprise 1 mg/mL to about 8 mg/mL citric acid. In some embodiments, the thin film may comprise between about 0.5 mg/mL and about 5 mg/mL tartaric acid. In some embodiments, the thin film may comprise about 0.05 mg/mL and about 0.75 mg/mL benzoic acid. In some embodiments, the thin film may comprise about 0.2% (v/v) to about 3% glycerol. In some embodiments, the thin film may comprise about 8 mg/mL to about 15 mg/mL L-lactic acid. In some embodiments, the thin film may comprise about 1 mg/mL to about 6 mg/mL sodium hydroxide. In some embodiments, the thin film may not comprise alginic acid.

In some embodiments, about 2.5 cm² of the thin film has a buffering capacity of at least about 0.3 mEq NaOH. In some embodiments, the thin film has a dispersion time in water of less than about 5 minutes. In some embodiments, the thin film has a dispersion time in water of less than about 4 minutes.

In some embodiments, the thin film has a bioadhesive strength of about 0.2 to about 1 N. In some embodiments, the thin film has a pH after dispersion in saline of less than about 3.7. In some embodiments, the thin film has a viscosity after dispersion in simulated vaginal fluid (SVF) of at least about 20 cP. In some embodiments, the thin film is stable for at least 6 months at 40° C. and 75% relative humidity (R.H.). In some embodiments, the thin film has a tensile strength of between about 0.8 Mpa and about 2 Mpa. In some embodiments, the thin film has a % elongation of between about 25 and 40. In some embodiments, the thin film has a toughness of between about 0.1 MJ/m³ and 0.35 MJ/m³.

In yet another aspect, the present disclosure provides a method of making a thin film, comprising providing an aqueous solution comprising L-lactic acid, a water-soluble polymer, and a plasticizer; casting the aqueous solution in a suitable substrate; and drying the aqueous solution to provide a thin film.

In yet another aspect, the present disclosure provides a method of making a thin film, comprising: providing an aqueous solution comprising PVA, HPMC, xanthan gum, citric acid, tartaric acid, benzoic acid, glycerol, L-lactic acid, and sodium hydroxide; casting the aqueous solution in a suitable substrate; and drying the aqueous solution to provide a thin film.

In some embodiments, the aqueous solution further comprises an additional buffering agent. In some embodiments, the additional buffering agent is an organic acid. In some embodiments, the additional buffering agent is selected from citric acid, tartaric acid, benzoic acid, alginic acid, sodium monocitrate, and any combination thereof.

In some embodiments, the method may further comprise a preservative. In some embodiments, the preservative is benzoic acid and/or sodium benzoate.

In some embodiments, the water-soluble polymer is selected from hydroxypropyl methylcellulose (HPMC), polyvinyl alcohol (PVA), xanthan gum, sodium alginate, carbopol, hydroxyethylcellulose, and any combination thereof. In some embodiments, the water-soluble polymer comprises HPMC. In some embodiments, the water-soluble polymer comprises PVA. In some embodiments, the water-soluble polymer comprises PVA and HPMC. In some embodiments, the water-soluble polymer comprises xanthan gum, PVA and HPMC. In some embodiments, the PVA has an average molecular weight between about 30,000 and about 186,000.

In some embodiments, the plasticizer is selected from glycerol, polyethylene glycol, propylene glycol, sorbitol, and any combination thereof. In some embodiments, the plasticizer is glycerol. In some embodiments, the glycerol is present in the aqueous solution at a concentration between about 0.2% (v/v) and about 4% (v/v).

In some embodiments, the L-lactic acid is present in the aqueous solution at a concentration between about 5 mg/mL and about 20 mg/mL. In some embodiments, the L-lactic acid is present at a concentration between about 8 mg/mL and about 15 mg/mL. In some embodiments, the water-soluble polymer is present at a concentration between about 1 mg/mL and about 150 mg/mL.

In some embodiments, the method may produce a thin film that does not comprise alginic acid.

In some embodiments, the aqueous solution comprises about 40 mg/mL to about 80 mg/mL PVA having an average molecular weight of about 30,000 to about 70,000; about 0 mg/mL to about 10 mg/mL PVA having an average molecular weight of about 85,000 to about 124,000; and about 0 mg/mL and about 8 mg/mL PVA having an average molecular weight of about 146,000 to about 186,000. In some embodiments, the aqueous solution comprises about 40 mg/mL to about 80 mg/mL PVA having an average molecular weight of about 30,000 to about 70,000 and about 2 mg/mL to about 10 mg/mL PVA having an average molecular weight of about 85,000 to about 124,000. In some embodiments, the aqueous solution comprises about 40 mg/mL to about 80 mg/mL PVA having an average molecular weight of about 30,000 to about 70,000 and about 1 mg/mL to about 10 mg/mL PVA having an average molecular weight of about 146,000 to about 186,000.

In some embodiments, the aqueous solution comprises about 1 mg/mL to about 10 mg/mL HPMC. In some embodiments, the HPMC is HPMC E50. In some embodiments, the HPMC is HPMC K15.

In some embodiments, the aqueous solution further comprises between about 0.1 mg/mL and about 1.5 mg/mL xanthan gum. In some embodiments, the aqueous solution further comprises 1 mg/mL to about 8 mg/mL citric acid. In some embodiments, the aqueous solution further comprises between about 0.5 mg/mL and about 5 mg/mL tartaric acid. In some embodiments, the aqueous solution further comprises about 0.05 mg/mL and about 0.75 mg/mL benzoic acid. In some embodiments, the aqueous solution further comprises about 0.2% (v/v) to about 3% glycerol. In some embodiments, the aqueous solution further comprises about 8 mg/mL to about 15 mg/mL L-lactic acid. In some embodiments, the aqueous solution further comprises about 1 mg/mL to about 6 mg/mL sodium hydroxide. In some embodiments, the aqueous solution does not comprise alginic acid.

In some embodiments, the method may further comprise removing the thin film from the substrate. In some embodiments, the method may further comprise de-aeration of the aqueous solution. In some embodiments, the method may further comprise cutting the film. In some embodiments, casting comprises pouring, extruding, or printing the aqueous solution onto the substrate.

In yet another aspect, the present disclosure provides a method of contraception, comprising intravaginally administering any of the thin films described herein, or any of the thin films made by the methods described herein.

In yet another aspect, the present disclosure provides a method of preventing transmission of a sexually transmitted infection, comprising intravaginally administering any of the thin films described herein, or the thins film made by any of the methods described herein.

In yet another aspect, the present disclosure provides a method of contraception and preventing a sexually transmitted infection, comprising intravaginally administering any of the thin films described herein, or the thin films made by any of the methods described herein.

In yet another aspect, the present disclosure provides a kit comprising a plurality of containers, each container comprising a thin film as described herein, or the thin film made by the methods described herein.

In some embodiments, the kit may further comprise instructions for use of the thin film as a contraceptive. In some embodiments, the kit may further comprise an applicator. In some embodiments, the applicator is configured to insert the thin film into a vaginal cavity.

### BRIEF DESCRIPTION OF THE DRAWINGS

FIG. 1 shows FTIR spectra of HPMC.

FIG. 2 shows FTIR spectra of Xanthan gum.

FIG. 3 shows FTIR spectra of HPMC and Xanthan gum mixture.

FIG. 4 shows FTIR spectra of HPMC, Xanthan gum, HPMC and Xanthan gum mixture, and HPMC and Xanthan gum film.

FIG. 5 shows the FTIR spectrum of alginic acid.

FIG. 6 shows FTIR spectra of Xanthan gum.

FIG. 7 shows the FTIR spectrum of Xanthan gum and alginic acid mixture.

FIG. 8 shows FTIR spectra of alginic acid, Xanthan gum, and a mixture of the two.

FIG. 9 shows photos of the blank vaginal films, as indicated.

FIG. 10 shows the buffering activity of film F20 against 1N sodium hydroxide.

FIG. 11 shows the buffering activity of film F21 against 1N sodium hydroxide.

FIGS. 12A-12C show results from testing various formulations for their properties. FIG. 12A shows tensile strength (TS), FIG. 12B shows % Elongation, and FIG. 12C shows toughness of various formulations.

FIG. 13 shows the buffering capacity of vaginal film F48 against 1N sodium hydroxide, compared to gel.

FIGS. 14A-14C are graphs showing the properties of formulations F48, F70, and F71 compared to vaginal contraceptive film (VCF). FIG. 14A shows tensile strength, FIG. 14B shows % Elongation, and FIG. 14C shows toughness of the various films.

FIG. 15 is a graph illustrating the buffering capacity of vaginal film F48, F68, F70, F71, and the gel composition against 1N sodium hydroxide.

FIG. 16 is a graph showing the buffering capacity of vaginal film F48, F70, F71, and the gel composition.

FIG. 17 is a graph showing the dispersion time of films F48, F70, F71, and VCF.

FIG. 18 shows FTIR spectra of the physical mixtures of acid buffering ingredients and film-forming polymers.

FIGS. 19A-19C are graphs showing characteristics of various films compared to VCF. FIG. 19A shows the tensile strength, FIG. 19B shows the % elongation, and FIG. 19C shows the toughness of the respective films compared to VCF.

FIG. 20 shows FTIR spectra of Film F70 before and after subjecting to high temperature at 50° C. for 1 week.

FIG. 21 shows FTIR spectra of Film F70 before and after subjecting the film to a high temperature at 50° C. for 1 week from 2000 to 450 cm⁻¹.

FIG. 22 shows FTIR spectra of Film F70 before and after subjecting film and physical mixture to high temperature at 50° C. for 1 week from 2000 to 450 cm⁻¹.

FIG. 23 is a graph showing the results of the mucoadhesion for preliminary films, the gel, and VCF.

FIG. 24 shows the dispersion of the film F70 in SVF.

FIG. 25 is a graph showing the acid buffering capacity of film F70.

FIG. 26 is a graph showing the DSC result of the newly fabricated film, and film subjected to heating at 50° C.

FIG. 27 is a graph showing DSC result of physical mixtures (PVA, HPMC, Xanthan gum, citric acid, tartaric acid, and benzoic acid).

FIG. 28 is a graph showing XRD of newly fabricated film, and film subjected to heating at 50° C.

FIG. 29 is a graph showing the viscosity for diluted gel composition sample PGH 50% dilution at 37° C.

FIG. 30 is a graph showing the viscosity of new composition film with different dilution fluid (SVF and saline).

FIGS. 31A-31D are graphs showing the stabilities of various properties of the film from time zero to 2 months. FIG. 31A shows the stability of tensile strength. FIG. 31B shows the stability of dispersion time in SVF. FIG. 31C shows the stability of acid bufferin capacity. FIG. 31D shows the stability of mucoadhesive strength.

FIGS. 32A-32C are graphs showing the stabilities of various properties of the film from time zero to 3 months. FIG. 32A shows the stability of tensile strength. FIG. 32B shows the stability of mucoadhesive strength. FIG. 32C shows the stability of acid buffering capacity.

FIGS. 33A-33D are graphs showing the stabilities of various properties of the film from time zero to 6 months. FIG. 33A shows the stability of the acid buffering capacity.

FIG. 33B shows the stability of the dispersion time. FIG. 33C shows the stability of the tensile strength. FIG. 33D shows the stability of mucoadhesive strength.

FIG. 34 is a summary of the physical properties of potential films compared with VCF.

FIG. 35 is a summary of the properties of film F70-1.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As used herein, the terms “antimicrobial,” “microbicide,” and “microbicidal” refer to a compound capable of preventing or inhibiting the growth and/or preventing or reducing the infectivity of microbes, including viruses, bacteria, fungi, protozoa, parasites, and algae.

As used herein, the term “sexually transmitted disease” is used interchangeably with “STD,” “sexually transmitted infection,” “STI” and/or the plural thereof. An STD is an illness or pathophysiological condition that has a significant probability of transmission between humans by means of any form of sexual contact, including kissing. The term STD may also encompass a person who is infected, and may potentially infect others, without showing signs of disease or infection.

The “buffering capacity” of the film is defined as the ability to maintain a desired pH when contacted with a compound having a different pH. In particular, and in the context of the present disclosure, buffering capacity means the ability to maintain a healthy vaginal pH. In some embodiments, buffering capacity is defined as the amount of sodium hydroxide that is required to raise the pH of the film (after dispersion into solution) from 3.5 to 5.

“Treating” and “treatment” as used herein include therapeutic and prophylactic treatment. Treatment methods include administering to a subject a therapeutically effective amount of an active agent. The administering step may consist of a single administration or may include a series of administrations. The length of the treatment period depends on a variety of factors, such as the severity of the risk or condition, the age of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the subject.

An “effective amount” is an amount sufficient for a composition to accomplish a stated purpose relative to the absence of the composition (e.g. achieve the effect for which it is administered). An example of an “effective amount” is an amount sufficient to contribute to preventing conception and/or preventing transmission of an STI. A “prophylactically effective amount” of a drug is an amount of the drug that, when administered to a subject, will have the intended prophylactic effect, e.g., preventing conception or transmission of an STI, or reducing the likelihood of conception or transmission of an STI. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Subject” or “subject in need thereof” refers to a living organism in need of contraception or prevention of disease or infection. Non-limiting examples include humans, other mammals, bovines, rats, mice, dogs, monkeys, goat, sheep, cows, deer, and other non-mammalian animals. In some embodiments, a patient is human. In some embodiments, the patient is a woman. However, the composition may be administered to any subject engaging in sexual contact, regardless of gender.

As used herein, the term “administering” means vaginal administration for example, by inserting the composition described herein onto or into the vaginal cavity, or to the lining of the vagina and/or cervix. Administering may also refer to rectal administration.

As used herein, the term “contacting” refers to any suitable method of bringing one or more of the compounds described herein into contact with a sperm and/or a sexually-transmitted or sexually-acquired microbe or microbial cell, etc., as described herein. In vitro or ex vivo, this is achieved by exposing the microbe or microbial cell to the microbicide in a suitable medium. For exemplary in vivo applications, topical methods of administration are suitable as described herein.

The term “basic solution” means a solution that has a pH above 7, such as 8, 9, 10, 11, 12, or 13. Exemplary bases for making a “basic solution” include, but are not limited to sodium hydroxide, potassium hydroxide, and lithium hydroxide.

As used herein, “saline” is a mixture of sodium chloride (salt) and water. Normal saline is the commonly used phrase for a solution of 0.90% w/v of NaCl, 308 mOsm/L or 9.0 g per liter. Less commonly, this solution is referred to as physiological saline or isotonic saline (because it is approximately isotonic to blood serum, which makes it a physiologically normal solution).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Compositions

In some embodiments, the present disclosure provides a thin film comprising L-lactic acid, a water-soluble polymer, and a plasticizer.

Lactic acid is produced by lactic acid bacteria such as Lactobacillus species. However, lactic acid bacteria generally produce both D and L lactic acid. Furthermore, lactic acid bacteria can be difficult to grow. Recombinant methods can be used to specifically manufacture L-lactic acid using hosts that are easier to grow, such as yeast or Escherichia coli (Ishida et al., Appl Environ Microbiol. 2005 April; 71 (4): 1964-1970 and Dien et al., J Ind Microbiol Biotechnol. 2001 October; 27 (4): 259-264). Alternatively, purified L-lactic acid can be purchased from established chemical suppliers such as Sigma-Aldrich® (St. Louis, Missouri). In specific embodiments, the lactic acid is L-lactic acid. In alternate embodiments, the lactic acid is racemic lactic acid.

Water-soluble polymers are substances that dissolve, disperse, or swell in water and, thus, modify the physical properties of aqueous systems in the form of gelation, thickening, or emulsification/stabilization. Water-soluble polymers may acquire tridimensional structures in an aqueous solution in a reversible way by promoting or increasing physical interactions between polymer chains. While precipitating and collapsing, macromolecules may capture cells and other biomolecules from an aqueous solution together with water molecules, thus creating hydrogel structures that contain active compounds. This feature is especially important for applications in biomedicine. There is an increased interest in this type of physically cross-linked nanostructured hydrogels due to their relative ease of production and the advantage of not having to use cross-linking agents. These agents can affect the integrity of substances to be entrapped (e.g., cells, proteins, etc.) and, consequently, sometimes they need to be removed before application, which is not an easy task.

In some embodiments, the water-soluble polymer may include, but is not necessarily limited to, hydroxypropyl methylcellulose (HPMC), polyvinyl alcohol (PVA), xanthan gum, sodium alginate, carbopol, hydroxyethylcellulose, and any combination thereof. In some embodiments, the water-soluble polymer is HPMC. In some embodiments, the water-soluble polymer is PVA. In some embodiments, the water-soluble polymer includes HPMC. In some embodiments, the water-soluble polymer includes PVA. In some embodiments, the water-soluble polymer includes xanthan gum. In some embodiments, the water-soluble polymer includes sodium alginate. In some embodiments, the water-soluble polymer includes carbopol. In some embodiments, the water-soluble polymer includes hydroxyethylcellulose. In some embodiments, the water-soluble polymer comprises PVA and HPMC. In some embodiments, the water-soluble polymer comprises xanthan gum, PVA and HPMC. In some embodiments, the PVA has an average molecular weight between about 30,000 and about 186,000.

In some embodiments, the water-soluble polymer is present at a concentration between about 1 mg/mL and about 150 mg/mL.

Plasticizers are substances that are added to a material to make it softer and more flexible, to increase its plasticity, to decrease its viscosity, or to decrease friction during its handling in manufacture. Plasticizers are commonly added to polymers such as plastics and rubber, either to facilitate the handling of the raw material during fabrication, or to meet the demands of the end product's application. For example, plasticizers are commonly added to polyvinyl chloride (PVC), which is otherwise hard and brittle, to make it soft and pliable; which makes it suitable for products such as vinyl flooring, clothing, bags, hoses, and electric wire coatings. Plasticizers for polymers are either liquids with low volatility or solids. Almost 90% of polymer plasticizers, most commonly phthalate esters, are used in PVC, giving this material improved flexibility and durability. The majority is used in films and cables.

In some embodiments, the plasticizer may include, but is not necessarily limited to, glycerol, polyethylene glycol, propylene glycol, sorbitol, and any combination thereof. In specific embodiments, the plasticizer is glycerol. In some embodiments, the glycerol is present at a concentration between about 0.2% (v/v) and about 4% (v/v).

In some embodiments, the thin film may comprise an additional buffering agent acts to maintain the pH of the vagina within its normal acidic range (i.e., a pH of less than about 5 and more preferably in the range of about 3.5 to about 4.5), even in the presence of normal amounts of ejaculate. Besides lactic acid, suitable buffering agents include, but are not limited to, for example, citric acid, potassium acid tartrate, benzoic acid, alginic acid, sorbic acid, fumaric acid, ascorbic acid, stearic acid, oleic acid, tartaric acid, potassium bitartrate, benzoic acid, edetic acid ethylenediamine tetraacetic acid (EDTA), acetic acid, malic acid, and the like. The acids may be added as free acids, hydrates, or pharmaceutically acceptable salts. Of course, the free acids can be converted to the corresponding salts in situ (i.e., within the vagina). In various exemplary embodiments, several buffering agents are included in the composition to provide increased buffering capacity. In embodiments, the buffering agent includes lactic acid (e.g., L-lactic acid). In embodiments, the buffering agent includes citric acid. In embodiments, the buffering agent includes potassium acid tartrate. In embodiments, the buffering agent includes benzoic acid. In embodiments, the buffering agent includes alginic acid. In embodiments, the buffering agent includes sorbic acid. In embodiments, the buffering agent includes fumaric acid. In embodiments, the buffering agent includes ascorbic acid. In embodiments, the buffering agent includes stearic acid. In embodiments, the buffering agent includes oleic acid. In embodiments, the buffering agent includes tartaric acid. In embodiments, the buffering agent includes potassium bitartrate. In embodiments, the buffering agent includes benzoic acid. In embodiments, the buffering agent includes EDTA. In embodiments, the buffering agent includes acetic acid. In embodiments, the buffering agent includes malic acid.

Suitable thickeners include, but are not limited to, for example, xanthan gum, hydroxypropyl cellulose, hydroxypropyl methyl cellulose, sodium carboxymethyl cellulose, chitosan, polycarbophil, carbopol, gellan gum, poloxamer, carrageenan, iota carrageenan, and the like. In embodiments, the thin film includes xanthan gum. In embodiments, the thin film includes hydroxypropyl cellulose. In embodiments, the thin film includes hydroxypropyl methyl cellulose. In embodiments, the thin film includes sodium carboxymethyl cellulose. In embodiments, the thin film includes chitosan. In embodiments, the thin film includes polycarbophil. In embodiments, the thin film includes carbopol. In embodiments, the thin film includes gellan gum. In embodiments, the thin film includes poloxamer. In embodiments, the thin film includes carrageenan. In embodiments, the thin film includes iota carrageenan.

Suitable humectants include, but are not limited to, for example, glycerol, polyethylene glycols, propylene glycols, sorbitol, triacetin, and the like. Glycerol may also act as a lubricant. In embodiments, the thin film includes glycerol. In embodiments, the thin film includes polyethylene glycols. In embodiments, the thin film includes propylene glycols. In embodiments, the thin film includes sorbitol. In embodiments, the thin film includes triacetin.

In some embodiments, the thin film composition may comprise an additional buffering agent. The additional buffering agent may include, but is not necessarily limited to, an organic acid. In some embodiments, the additional buffering agent may include, but is not necessarily limited to, citric acid, tartaric acid, benzoic acid, alginic acid, sodium monocitrate, and any combination thereof.

Additionally, the thin film composition may also include a preservative. Suitable preservatives include, but are not limited to, for example, benzoic acid, sodium benzoate, methylparaben, ethylparaben, butylparaben, propylparaben, benzyalkonium chloride, phenylmercuric nitrate, chlorhexidine, and the like. In one exemplary embodiment, benzoic acid is used and may also contribute to the buffering capacity of the thin film composition. In specific embodiments, the preservative is benzoic acid and/or sodium benzoate. In embodiments, the preservative includes benzoic acid. In embodiments, the preservative includes sodium benzoate. In embodiments, the preservative includes methylparaben. In embodiments, the preservative includes ethylparaben. In embodiments, the preservative includes butylparaben. In embodiments, the preservative includes propylparaben. In embodiments, the preservative includes benzyalkonium chloride. In embodiments, the preservative includes phenylmercuric nitrate. In embodiments, the preservative includes chlorhexidine.

In some embodiments, the present disclosure provides a thin film composition comprising PVA, HPMC, xanthan gum, citric acid, tartaric acid, benzoic acid, glycerol, L-lactic acid, and sodium hydroxide. In some embodiments, the present disclosure provides a thin film composition consisting of or consisting essentially of PVA, HPMC, xanthan gum, citric acid, tartaric acid, benzoic acid, glycerol, L-lactic acid, and sodium hydroxide.

In some embodiments, the PVA in the thin film composition has an average molecular weight between about 20,000 and about 200,000. In some embodiments, the PVA in the thin film composition has an average molecular weight between about 30,000 and about 186,000. In some embodiments, the PVA in the thin film composition has an average molecular weight between about 30,000 and about 150,000. In some embodiments, the PVA in the thin film composition has an average molecular weight between about 30,000 and about 100,000. In some embodiments, the PVA has an average molecular weight between about 30,000 and about 70,000. In some embodiments, the PVA has an average molecular weight between about 50,000 and about 150,000. In some embodiments, the PVA has an average molecular weight between about 50,000 and about 124,000. In some embodiments, the PVA has an average molecular weight between about 85,000 and about 124,000. In some embodiments, the PVA has an average molecular weight between about 50,000 and about 200,000. In some embodiments, the PVA has an average molecular weight between about 75,000 and about 200,000. In some embodiments, the PVA has an average molecular weight between about 100,000 and about 200,000. In some embodiments, the PVA has an average molecular weight between about 120,000 and about 200,000. In some embodiments, the PVA has an average molecular weight between about 140,000 and about 200,000. In some embodiments, the PVA in the thin film composition has an average molecular weight between about 146,000 and about 200,000. In some embodiments, the PVA in the thin film composition has an average molecular weight between about 100,000 and about 186,000. In some embodiments, the PVA in the thin film composition has an average molecular weight between about 146,000 and about 186,000. The molecular weight may be any value or subrange within the recited ranges, including endpoints.

In some embodiments, the thin film compositions described herein may comprise about 10% (w/w) to about 40% (w/w) PVA having an average molecular weight of about 20,000 to about 75,000. In some embodiments, the thin film compositions described herein may comprise about 10% (w/w) to about 30% (w/w) PVA having an average molecular weight of about 20,000 to about 75,000. In some embodiments, the thin film compositions described herein may comprise about 10% (w/w) to about 25% (w/w) PVA having an average molecular weight of about 20,000 to about 75,000. In some embodiments, the thin film compositions described herein may comprise about 15% (w/w) to about 40% (w/w) PVA having an average molecular weight of about 20,000 to about 75,000. In some embodiments, the thin film compositions described herein may comprise about 20% (w/w) to about 40% (w/w) PVA having an average molecular weight of about 20,000 to about 75,000. In some embodiments, the thin film compositions described herein may comprise about 15% (w/w) to about 30% (w/w) PVA having an average molecular weight of about 20,000 to about 75,000. In some embodiments, the thin film compositions described herein may comprise about 15% (w/w) to about 25% (w/w) PVA having an average molecular weight of about 20,000 to about 75,000. In some embodiments, the thin film compositions described herein may comprise about 20% (w/w) to about 30% (w/w) PVA having an average molecular weight of about 20,000 to about 75,000. In some embodiments, the thin film compositions described herein may comprise about 20% (w/w) to about 25% (w/w) PVA having an average molecular weight of about 20,000 to about 75,000. The concentration and/or molecular weight may be any value or subrange within the recited ranges, including endpoints.

In some embodiments, the thin film compositions described herein may comprise about 10% (w/w) to about 40% (w/w) PVA having an average molecular weight of about 30,000 to about 70,000. In some embodiments, the thin film compositions described herein may comprise about 10% (w/w) to about 30% (w/w) PVA having an average molecular weight of about 30,000 to about 70,000. In some embodiments, the thin film compositions described herein may comprise about 10% (w/w) to about 25% (w/w) PVA having an average molecular weight of about 30,000 to about 70,000. In some embodiments, the thin film compositions described herein may comprise about 15% (w/w) to about 40% (w/w) PVA having an average molecular weight of about 30,000 to about 70,000. In some embodiments, the thin film compositions described herein may comprise about 20% (w/w) to about 40% (w/w) PVA having an average molecular weight of about 30,000 to about 70,000. In some embodiments, the thin film compositions described herein may comprise about 15% (w/w) to about 30% (w/w) PVA having an average molecular weight of about 30,000 to about 70,000. In some embodiments, the thin film compositions described herein may comprise about 15% (w/w) to about 25% (w/w) PVA having an average molecular weight of about 30,000 to about 70,000. In some embodiments, the thin film compositions described herein may comprise about 20% (w/w) to about 30% (w/w) PVA having an average molecular weight of about 30,000 to about 70,000. In some embodiments, the thin film compositions described herein may comprise about 20% (w/w) to about 25% (w/w) PVA having an average molecular weight of about 30,000 to about 70,000. The concentration and/or molecular weight may be any value or subrange within the recited ranges, including endpoints.

In some embodiments, the thin film compositions described herein may comprise about 0% (w/w) to about 10% (w/w) PVA having an average molecular weight of about 75,000 to about 140,000. In some embodiments, the thin film compositions described herein may comprise about 0% (w/w) to about 8% (w/w) PVA having an average molecular weight of about 75,000 to about 140,000. In some embodiments, the thin film compositions described herein may comprise about 0% (w/w) to about 6% (w/w) PVA having an average molecular weight of about 75,000 to about 140,000. In some embodiments, the thin film compositions described herein may comprise about 0% (w/w) to about 5% (w/w) PVA having an average molecular weight of about 75,000 to about 140,000. In some embodiments, the thin film compositions described herein may comprise about 0% (w/w) to about 4% (w/w) PVA having an average molecular weight of about 75,000 to about 140,000. In some embodiments, the thin film compositions described herein may comprise about 0% (w/w) to about 3% (w/w) PVA having an average molecular weight of about 75,000 to about 140,000. In some embodiments, the thin film compositions described herein may comprise less than about 5% (w/w) PVA having an average molecular weight of about 75,000 to about 140,000. In some embodiments, the thin film compositions described herein may comprise less than about 3% (w/w) PVA having an average molecular weight of about 75,000 to about 140,000. In some embodiments, the thin film compositions described herein may comprise about 1% (w/w) to about 10% (w/w) PVA having an average molecular weight of about 75,000 to about 140,000. In some embodiments, the thin film compositions described herein may comprise about 1% (w/w) to about 8% (w/w) PVA having an average molecular weight of about 75,000 to about 140,000. In some embodiments, the thin film compositions described herein may comprise about 1% (w/w) to about 6% (w/w) PVA having an average molecular weight of about 75,000 to about 140,000. In some embodiments, the thin film compositions described herein may comprise about 1% (w/w) to about 5% (w/w) PVA having an average molecular weight of about 75,000 to about 140,000. In some embodiments, the thin film compositions described herein may comprise about 1% (w/w) to about 4% (w/w) PVA having an average molecular weight of about 75,000 to about 140,000. In some embodiments, the thin film compositions described herein may comprise about 1% (w/w) to about 3% (w/w) PVA having an average molecular weight of about 75,000 to about 140,000. The concentration and/or molecular weight may be any value or subrange within the recited ranges, including endpoints.

In some embodiments, the thin film compositions described herein may comprise about 0% (w/w) to about 10% (w/w) PVA having an average molecular weight of about 85,000 to about 124,000. In some embodiments, the thin film compositions described herein may comprise about 0% (w/w) to about 8% (w/w) PVA having an average molecular weight of about 85,000 to about 124,000. In some embodiments, the thin film compositions described herein may comprise about 0% (w/w) to about 6% (w/w) PVA having an average molecular weight of about 85,000 to about 124,000. In some embodiments, the thin film compositions described herein may comprise about 0% (w/w) to about 5% (w/w) PVA having an average molecular weight of about 85,000 to about 124,000. In some embodiments, the thin film compositions described herein may comprise about 0% (w/w) to about 4% (w/w) PVA having an average molecular weight of about 85,000 to about 124,000. In some embodiments, the thin film compositions described herein may comprise about 0% (w/w) to about 3% (w/w) PVA having an average molecular weight of about 85,000 to about 124,000. In some embodiments, the thin film compositions described herein may comprise less than about 5% (w/w) PVA having an average molecular weight of about 85,000 to about 124,000. In some embodiments, the thin film compositions described herein may comprise less than about 3% (w/w) PVA having an average molecular weight of about 85,000 to about 124,000. In some embodiments, the thin film compositions described herein may comprise about 1% (w/w) to about 10% (w/w) PVA having an average molecular weight of about 85,000 to about 124,000. In some embodiments, the thin film compositions described herein may comprise about 1% (w/w) to about 8% (w/w) PVA having an average molecular weight of about 85,000 to about 124,000. In some embodiments, the thin film compositions described herein may comprise about 1% (w/w) to about 6% (w/w) PVA having an average molecular weight of about 85,000 to about 124,000. In some embodiments, the thin film compositions described herein may comprise about 1% (w/w) to about 5% (w/w) PVA having an average molecular weight of about 85,000 to about 124,000. In some embodiments, the thin film compositions described herein may comprise about 1% (w/w) to about 4% (w/w) PVA having an average molecular weight of about 85,000 to about 124,000. In some embodiments, the thin film compositions described herein may comprise about 1% (w/w) to about 3% (w/w) PVA having an average molecular weight of about 85,000 to about 124,000. The concentration and/or molecular weight may be any value or subrange within the recited ranges, including endpoints.

In some embodiments, the thin film compositions described herein may comprise about 0% (w/w) to about 10% (w/w) PVA having an average molecular weight of about 140,000 to about 200,000. In some embodiments, the thin film compositions described herein may comprise about 0% (w/w) to about 8% (w/w) PVA having an average molecular weight of about 140,000 to about 200,000. In some embodiments, the thin film compositions described herein may comprise about 0% (w/w) to about 6% (w/w) PVA having an average molecular weight of about 140,000 to about 200,000. In some embodiments, the thin film compositions described herein may comprise about 0% (w/w) to about 5% (w/w) PVA having an average molecular weight of about 140,000 to about 200,000. In some embodiments, the thin film compositions described herein may comprise about 0% (w/w) to about 4% (w/w) PVA having an average molecular weight of about 140,000 to about 200,000. In some embodiments, the thin film compositions described herein may comprise about 0% (w/w) to about 3% (w/w) PVA having an average molecular weight of about 140,000 to about 200,000. In some embodiments, the thin film compositions described herein may comprise less than about 5% (w/w) PVA having an average molecular weight of about 140,000 to about 200,000. In some embodiments, the thin film compositions described herein may comprise less than about 3% (w/w) PVA having an average molecular weight of about 140,000 to about 200,000. In some embodiments, the thin film compositions described herein may comprise about 1% (w/w) to about 10% (w/w) PVA having an average molecular weight of about 140,000 to about 200,000. In some embodiments, the thin film compositions described herein may comprise about 1% (w/w) to about 8% (w/w) PVA having an average molecular weight of about 140,000 to about 200,000. In some embodiments, the thin film compositions described herein may comprise about 1% (w/w) to about 6% (w/w) PVA having an average molecular weight of about 140,000 to about 200,000. In some embodiments, the thin film compositions described herein may comprise about 1% (w/w) to about 5% (w/w) PVA having an average molecular weight of about 140,000 to about 200,000. In some embodiments, the thin film compositions described herein may comprise about 1% (w/w) to about 4% (w/w) PVA having an average molecular weight of about 140,000 to about 200,000. In some embodiments, the thin film compositions described herein may comprise about 1% (w/w) to about 3% (w/w) PVA having an average molecular weight of about 140,000 to about 200,000. The concentration and/or molecular weight may be any value or subrange within the recited ranges, including endpoints.

In some embodiments, the thin film compositions described herein may comprise about 0% (w/w) to about 10% (w/w) PVA having an average molecular weight of about 146,000 to about 186,000. In some embodiments, the thin film compositions described herein may comprise about 0% (w/w) to about 8% (w/w) PVA having an average molecular weight of about 146,000 to about 186,000. In some embodiments, the thin film compositions described herein may comprise about 0% (w/w) to about 6% (w/w) PVA having an average molecular weight of about 146,000 to about 186,000. In some embodiments, the thin film compositions described herein may comprise about 0% (w/w) to about 5% (w/w) PVA having an average molecular weight of about 146,000 to about 186,000. In some embodiments, the thin film compositions described herein may comprise about 0% (w/w) to about 4% (w/w) PVA having an average molecular weight of about 146,000 to about 186,000. In some embodiments, the thin film compositions described herein may comprise about 0% (w/w) to about 3% (w/w) PVA having an average molecular weight of about 146,000 to about 186,000. In some embodiments, the thin film compositions described herein may comprise less than about 5% (w/w) PVA having an average molecular weight of about 146,000 to about 186,000. In some embodiments, the thin film compositions described herein may comprise less than about 3% (w/w) PVA having an average molecular weight of about 146,000 to about 186,000. In some embodiments, the thin film compositions described herein may comprise about 1% (w/w) to about 10% (w/w) PVA having an average molecular weight of about 146,000 to about 186,000. In some embodiments, the thin film compositions described herein may comprise about 1% (w/w) to about 8% (w/w) PVA having an average molecular weight of about 146,000 to about 186,000. In some embodiments, the thin film compositions described herein may comprise about 1% (w/w) to about 6% (w/w) PVA having an average molecular weight of about 146,000 to about 186,000. In some embodiments, the thin film compositions described herein may comprise about 1% (w/w) to about 5% (w/w) PVA having an average molecular weight of about 146,000 to about 186,000. In some embodiments, the thin film compositions described herein may comprise about 1% (w/w) to about 4% (w/w) PVA having an average molecular weight of about 146,000 to about 186,000. In some embodiments, the thin film compositions described herein may comprise about 1% (w/w) to about 3% (w/w) PVA having an average molecular weight of about 146,000 to about 186,000. The concentration and/or molecular weight may be any value or subrange within the recited ranges, including endpoints.

In some embodiments, the thin film compositions described herein may comprise about 10% (w/w) to about 40% (w/w) PVA having an average molecular weight of about 20,000 to about 75,000; about 0% (w/w) to about 10% (w/w) PVA having an average molecular weight of about 75,000 to about 140,000; and about 0% (w/w) to about 10% (w/w) PVA having an average molecular weight of about 140,000 to about 200,000. In some embodiments, the thin film compositions described herein may comprise about 10% (w/w) to about 40% (w/w) PVA having an average molecular weight of about 30,000 to about 70,000; about 0% (w/w) to about 10% (w/w) mg/mL PVA having an average molecular weight of about 85,000 to about 124,000; and about 0% (w/w) to about 10% PVA having an average molecular weight of about 146,000 to about 186,000. In some embodiments, the thin film compositions described herein may comprise about 10% (w/w) to about 25% (w/w) PVA having an average molecular weight of about 30,000 to about 70,000 and about 1% (w/w) to about 5% (w/w) PVA having an average molecular weight of about 85,000 to about 124,000. In some embodiments, the thin film compositions described herein may comprise about 10% (w/w) to about 25% (w/w) PVA having an average molecular weight of about 30,000 to about 70,000 and about 1% (w/w) to about 5% (w/w) PVA having an average molecular weight of about 146,000 to about 186,000. Concentrations or molecular weight may be any value or subrange within the recited ranges, including endpoints.

In some embodiments, the thin film compositions described herein may comprise about 0.5% (w/w) to about 10% (w/w) HPMC. In some embodiments, the thin film compositions described herein may comprise about 0.5% (w/w) to about 5% (w/w) HPMC. In some embodiments, the thin film compositions described herein may comprise about 0.5% (w/w) to about 2% (w/w) HPMC. In some embodiments, the thin film compositions described herein may comprise about 1% (w/w) to about 10% (w/w) HPMC. In some embodiments, the thin film compositions described herein may comprise about 1% (w/w) to about 8% (w/w) HPMC. In some embodiments, the thin film compositions described herein may comprise about 1% (w/w) to about 6% (w/w) HPMC. In some embodiments, the thin film compositions described herein may comprise about 1% (w/w) to about 5% (w/w) HPMC. In some embodiments, the thin film compositions described herein may comprise about 1% (w/w) to about 4% (w/w) HPMC. In some embodiments, the thin film compositions described herein may comprise about 1% (w/w) to about 3% (w/w) HPMC. In some embodiments, the thin film compositions described herein may comprise about 1% (w/w) to about 2% (w/w) HPMC. In some embodiments, the thin film compositions described herein may comprise less than about 2% (w/w) HPMC. In some embodiments, the thin film compositions described herein may comprise about 1.8% (w/w) HPMC. Concentration may be any value or subrange within the recited ranges, including endpoints.

In specific embodiments, the HPMC is HPMC E50. In specific embodiments, the HPMC is HPMC K15.

In some embodiments, the thin compositions described herein may comprise between about 0.05% (w/w) to about 1.5% (w/w) xanthan gum. In some embodiments, the thin compositions described herein may comprise between about 0.1% (w/w) to about 1.5% (w/w) xanthan gum. In some embodiments, the thin compositions described herein may comprise between about 0.2% (w/w) to about 1.5% (w/w) xanthan gum. In some embodiments, the thin compositions described herein may comprise between about 0.1% (w/w) to about 1.0% (w/w) xanthan gum. In some embodiments, the thin compositions described herein may comprise between about 0.1% (w/w) to about 0.5% (w/w) xanthan gum. In some embodiments, the thin compositions described herein may comprise between about 0.1% (w/w) to about 0.4% (w/w) xanthan gum. In some embodiments, the thin compositions described herein may comprise between about 0.1% (w/w) to about 0.3% (w/w) xanthan gum. Concentration may be any value or subrange within the recited ranges, including endpoints.

In some embodiments, the thin film compositions described herein may comprise between about 10% (w/w) and about 20% (w/w) citric acid. In some embodiments, the thin film compositions described herein may comprise between about 12% (w/w) and about 20% (w/w) citric acid. In some embodiments, the thin film compositions described herein may comprise between about 10% (w/w) and about 15% (w/w) citric acid. In some embodiments, the thin film compositions described herein may comprise between about 12% (w/w) and about 15% (w/w) citric acid. Concentration may be any value or subrange within the recited ranges, including endpoints.

In some embodiments, the thin film compositions described herein may comprise between about 4% (w/w) and about 15% (w/w) tartaric acid. In some embodiments, the thin film compositions described herein may comprise between about 6% (w/w) and about 15% (w/w) tartaric acid. In some embodiments, the thin film compositions described herein may comprise between about 8% (w/w) and about 15% (w/w) tartaric acid. In some embodiments, the thin film compositions described herein may comprise between about 4% (w/w) and about 10% (w/w) tartaric acid. In some embodiments, the thin film compositions described herein may comprise between about 6% (w/w) and about 10% (w/w) tartaric acid. In some embodiments, the thin film compositions described herein may comprise between about 8% (w/w) and about 10% (w/w) tartaric acid. Concentration may be any value or subrange within the recited ranges, including endpoints.

In some embodiments, the thin film compositions described herein may comprise about 0.2% (w/w) and about 2% (w/w) benzoic acid. In some embodiments, the thin film compositions described herein may comprise about 0.5% (w/w) and about 2% (w/w) benzoic acid. In some embodiments, the thin film compositions described herein may comprise about 0.2% (w/w) and about 1.5% (w/w) benzoic acid. In some embodiments, the thin film compositions described herein may comprise about 0.2% (w/w) and about 1% (w/w) benzoic acid. In some embodiments, the thin film compositions described herein may comprise about 0.5% (w/w) and about 1.5% (w/w) benzoic acid. In some embodiments, the thin film compositions described herein may comprise about 0.5% (w/w) and about 1% (w/w) benzoic acid. Concentration may be any value or subrange within the recited ranges, including endpoints.

In some embodiments, the thin film compositions described herein may comprise about 5% (w/w) to about 15% (w/w) glycerol. In some embodiments, the thin film compositions described herein may comprise about 5% (w/w) to about 12% (w/w) glycerol. In some embodiments, the thin film compositions described herein may comprise about 5% (w/w) to about 10% (w/w) glycerol. In some embodiments, the thin film compositions described herein may comprise about 7% (w/w) to about 15% (w/w) glycerol. In some embodiments, the thin film compositions described herein may comprise about 8% (w/w) to about 15% (w/w) glycerol. In some embodiments, the thin film compositions described herein may comprise about 8% (w/w) to about 12% (w/w) glycerol. In some embodiments, the thin film compositions described herein may comprise about 8% (w/w) to about 10% (w/w) glycerol. Concentration may be any value or subrange within the recited ranges, including endpoints.

In some embodiments, the thin film compositions described herein may comprise about 10% (w/w) to about 50% (w/w) L-lactic acid. In some embodiments, the thin film compositions described herein may comprise about 20% (w/w) to about 50% (w/w) L-lactic acid. In some embodiments, the thin film compositions described herein may comprise about 25% (w/w) to about 50% (w/w) L-lactic acid. In some embodiments, the thin film compositions described herein may comprise about 10% (w/w) to about 40% (w/w) L-lactic acid. In some embodiments, the thin film compositions described herein may comprise about 10% (w/w) to about 35% (w/w) L-lactic acid. In some embodiments, the thin film compositions described herein may comprise about 10% (w/w) to about 30% (w/w) L-lactic acid. In some embodiments, the thin film compositions described herein may comprise about 20% (w/w) to about 40% (w/w) L-lactic acid. In some embodiments, the thin film compositions described herein may comprise about 20% (w/w) to about 30% (w/w) L-lactic acid. Concentration may be any value or subrange within the recited ranges, including endpoints.

In some embodiments, the thin film compositions described herein may comprise about 8% (w/w) to about 20% (w/w) sodium hydroxide. In some embodiments, the thin film compositions described herein may comprise about 8% (w/w) to about 15% (w/w) sodium hydroxide. In some embodiments, the thin film compositions described herein may comprise about 10% (w/w) to about 20% (w/w) sodium hydroxide. In some embodiments, the thin film compositions described herein may comprise about 8% (w/w) to about 15% (w/w) sodium hydroxide. In some embodiments, the thin film compositions described herein may comprise about 12% (w/w) to about 20% (w/w) sodium hydroxide. In some embodiments, the thin film compositions described herein may comprise about 12% (w/w) to about 15% (w/w) sodium hydroxide. Concentration may be any value or subrange within the recited ranges, including endpoints.

In some embodiments, the thin film compositions described herein do not include alginic acid.

In embodiments, the thin film comprises about 45% to about 60% PVA, about 4% to about 5% HPMC, about 0.5% to about 1% xanthan gum, about 18% to about 22% glycerol, about 10% to about 14% lactic acid, and about 4% to about 11% additional buffering agent. In embodiments, the thin film consists essentially of about 45% to about 60% PVA, about 4% to about 5% HPMC, about 0.5% to about 1% xanthan gum, about 18% to about 22% glycerol, about 10% to about 14% lactic acid, and about 4% to about 11% additional buffering agent. In embodiments, the thin film consists of about 45% to about 60% PVA, about 4% to about 5% HPMC, about 0.5% to about 1% xanthan gum, about 18% to about 22% glycerol, about 10% to about 14% lactic acid, and about 4% to about 11% additional buffering agent.

In embodiments, the PVA comprises PVA with a molecular weight (MW) between about 30,000 to about 70,000; between about 85,000 to about 124,000; and/or between about 146,000 to about 186,000. In embodiments, the PVA comprises about 45% to about 60% of PVA with a MW between about 30,000 to about 70,000, about 0% to about 8% of PVA with a MW between about 85,000 to about 124,000, and about 0% to about 6% of PVA with a MW of about 146,000 to about 186,000.

In embodiments, the additional buffering agent comprises citric acid, tartaric acid, benzoic acid, and/or sodium hydroxide. In embodiments, the citric acid is at a concentration of about 2.5% to about 4%. In embodiments, the tartaric acid is at a concentration of about 1.5% to about 3%. In embodiments, the benzoic acid is at a concentration of about 0.1% to about 0.4%. In embodiments, the sodium hydroxide is at a concentration of about 2% to about 4%. In embodiments, the citric acid is at a concentration of about 2.5% to about 4%, the tartaric acid is at a concentration of about 1.5% to about 3%, the benzoic acid is at a concentration of about 0.1% to about 0.4%, and the sodium hydroxide is at a concentration of about 2% to about 4%.

In some embodiments, about 2.5 cm² of the thin films described herein have a buffering capacity of at least about 0.2 mEq NaOH. In some embodiments, about 2.5 cm² of the thin films described herein have a buffering capacity of at least about 0.25 mEq NaOH. In some embodiments, about 2.5 cm² of the thin films described herein have a buffering capacity of at least about 0.3 mEq NaOH. In some embodiments, about 2.5 cm² of the thin films described herein have a buffering capacity of at least about 0.35 mEq NaOH. In some embodiments, about 2.5 cm² of the thin films described herein have a buffering capacity of at least about 0.4 mEq NaOH. In some embodiments, about 2.5 cm² of the thin films described herein have a buffering capacity of at least about 0.45 mEq NaOH. The buffering capacity may be any value or subrange within the recited values or ranges, including endpoints.

In some embodiments, the thin films described herein have a dispersion time in water of less than about 5 minutes. In some embodiments, the thin films have a dispersion time in water of less than about 4.5 minutes. In some embodiments, the thin films have a dispersion time in water of less than about 4 minutes. In some embodiments, the thin films have a dispersion time in water of less than about 3.5 minutes. In some embodiments, the thin films have a dispersion time in water of less than about 3 minutes. In some embodiments, the thin films have a dispersion time in water of less than about 2.5 minutes. The dispersion time may be any value or subrange within the recited ranges or values, including endpoints.

In some embodiments, the thin films described herein have a bioadhesive strength of about 0.2 N to about 1 N. In some embodiments, the thin films described herein have a bioadhesive strength of about 0.4 N to about 1 N. In some embodiments, the thin films described herein have a bioadhesive strength of about 0.5 N to about 1 N. In some embodiments, the thin films described herein have a bioadhesive strength of about 0.75 N to about 1 N. In some embodiments, the thin films described herein have a bioadhesive strength of about 0.2 N to about 0.8 N. In some embodiments, the thin films described herein have a bioadhesive strength of about 0.4 N to about 0.8 N. In some embodiments, the thin films described herein have a bioadhesive strength of about 0.5 N to about 0.8 N. The strength may be any value or subrange within the recited ranges, including endpoints.

In some embodiments, the thin films described herein have a pH after dispersion in saline of less than about 5. In some embodiments, the thin films described herein have a pH after dispersion in saline of less than about 4. In some embodiments, the thin films described herein have a pH after dispersion in saline of less than about 3.9. In some embodiments, the thin films described herein have a pH after dispersion in saline of less than about 3.8. In some embodiments, the thin films described herein have a pH after dispersion in saline of less than about 3.7. In some embodiments, the thin films described herein have a pH after dispersion in saline of less than about 3.6. In some embodiments, the thin films described herein have a pH after dispersion in saline of less than about 3.5. In some embodiments, the thin films described herein have a pH after dispersion in saline of less than about 3.4. In some embodiments, the thin films described herein have a pH after dispersion in saline of less than about 3.3. The pH may be any value or subrange within the recited ranges or values, including endpoints.

In some embodiments, the thin films described herein have a pH after dispersion in simulated vaginal fluid (SVF) of less than about 5. In some embodiments, the thin films described herein have a pH after dispersion in simulated vaginal fluid (SVF) of less than about 4. In some embodiments, the thin films described herein have a pH after dispersion in SVF of less than about 3.9. In some embodiments, the thin films described herein have a pH after dispersion in SVF of less than about 3.8. In some embodiments, the thin films described herein have a pH after dispersion in SVF of less than about 3.7. In some embodiments, the thin films described herein have a pH after dispersion in SVF of less than about 3.6. In some embodiments, the thin films described herein have a pH after dispersion in SVF of less than about 3.5. In some embodiments, the thin films described herein have a pH after dispersion in SVF of less than about 3.4. In some embodiments, the thin films described herein have a pH after dispersion in SVF of less than about 3.3. The pH may be any value or subrange within the recited ranges or values, including endpoints.

In some embodiments, the thin films described herein have a viscosity after dispersion in SVF of at least about 20 cP. In some embodiments, the thin films described herein have a viscosity after dispersion in SVF of at least about 30 cP. In some embodiments, the thin films described herein have a viscosity after dispersion in SVF of at least about 40 cP. In some embodiments, the thin films described herein have a viscosity after dispersion in SVF of at least about 50 cP. In some embodiments, the thin films described herein have a viscosity after dispersion in SVF of at least about 100 cP. The pH may be any value or subrange within the recited ranges or values, including endpoints.

In some embodiments, the thin films described herein are stable for at least 2 months at 40° C. and 75% relative humidity. In some embodiments, the thin films described herein are stable for at least 3 months at 40° C. and 75% relative humidity. In some embodiments, the thin films described herein are stable for at least 4 months at 40° C. and 75% relative humidity. In some embodiments, the thin films described herein are stable for at least 5 months at 40° C. and 75% relative humidity. In some embodiments, the thin films described herein are stable for at least 6 months at 40° C. and 75% relative humidity. In some embodiments, the thin films described herein are stable for at least 8 months at 40° C. and 75% relative humidity. In some embodiments, the thin films described herein are stable for at least 10 months at 40° C. and 75% relative humidity. In some embodiments, the thin films described herein are stable for at least 12 months at 40° C. and 75% relative humidity.

In some embodiments, the thin films described herein are stable for at least 2 months at 25° C. and 6% relative humidity. In some embodiments, the thin films described herein are stable for at least 3 months at 25° C. and 6% relative humidity. In some embodiments, the thin films described herein are stable for at least 4 months at 25° C. and 6% relative humidity. In some embodiments, the thin films described herein are stable for at least 5 months at 25° C. and 6% relative humidity. In some embodiments, the thin films described herein are stable for at least 6 months at 25° C. and 6% relative humidity. In some embodiments, the thin films described herein are stable for at least 8 months at 25° C. and 6% relative humidity. In some embodiments, the thin films described herein are stable for at least 10 months at 25° C. and 6% relative humidity. In some embodiments, the thin films described herein are stable for at least 12 months at 25° C. and 6% relative humidity.

Stability refers to no change or minimal change on one or more physiochemical parameters, for example appearance, acid buffering capacity, dispersion time, moisture content (e.g., measured by change in weight of the film over time), viscosity after dispersion, and mechanical strength. In embodiments, the film is stable if the indicated parameter does not change by more than about 30% under the indicated conditions. In embodiments, the film is stable if the indicated parameter does not change by more than about 25% under the indicated conditions. In embodiments, the film is stable if the indicated parameter does not change by more than about 30% under the indicated conditions. In embodiments, the film is stable if the indicated parameter does not change by more than about 20% under the indicated conditions. In embodiments, the film is stable if the indicated parameter does not change by more than about 15% under the indicated conditions. In embodiments, the film is stable if the indicated parameter does not change by more than about 10% under the indicated conditions. In embodiments, the film is stable if the indicated parameter does not change by more than about 5% under the indicated conditions. In embodiments, the film is stable if the indicated parameter does not change by more than about 1% under the indicated conditions.

In some embodiments, the thin films described herein have a tensile strength of between about 0.5 Mpa and about 2 Mpa. In some embodiments, the thin films described herein have a tensile strength of between about 0.8 Mpa and about 2 Mpa. In some embodiments, the thin films described herein have a tensile strength of between about 0.9 Mpa and about 2 Mpa. In some embodiments, the thin films described herein have a tensile strength of between about 1 Mpa and about 2 Mpa. In some embodiments, the thin films described herein have a tensile strength of between about 0.8 Mpa and about 1.5 Mpa. In some embodiments, the thin films described herein have a tensile strength of between about 0.8 Mpa and about 1.3 Mpa. The tensile strength may be any value or subrange within the recited ranges, including endpoints.

In some embodiments, the thin films described herein have a % elongation of between about 20 and about 50. In some embodiments, the thin films described herein have a % elongation of between about 25 and about 50. In some embodiments, the thin films described herein have a % elongation of between about 25 and about 45. In some embodiments, the thin films described herein have a % elongation of between about 25 and about 40. In some embodiments, the thin films described herein have a % elongation of between about 25 and about 35. The % elongation may be any value or subrange within the recited ranges, including endpoints.

In some embodiments, the thin films described herein have a toughness of between about 0.06 MJ/m³ and about 0.5 MJ/m³. In some embodiments, the thin films described herein have a toughness of between about 0.06 MJ/m³ and about 0.35 MJ/m³. In some embodiments, the thin films described herein have a toughness of between about 0.08 MJ/m³ and about 0.35 MJ/m³. In some embodiments, the thin films described herein have a toughness of between about 0.1 MJ/m³ and about 0.35 MJ/m³. In some embodiments, the thin films described herein have a toughness of between about 0.1 MJ/m³ and about 0.3 MJ/m³. In some embodiments, the thin films described herein have a toughness of between about 0.1 MJ/m³ and about 0.25 MJ/m³. In some embodiments, the thin films described herein have a toughness of between about 0.15 MJ/m³ and about 0.35 MJ/m³. The toughness may be any value or subrange within the recited ranges, including endpoints.

In some embodiments, the thin film may comprise one or more additional pharmaceutically acceptable excipients.

### Methods

In some embodiments, the present disclosure provides a method of making a thin film, comprising providing an aqueous solution comprising L-lactic acid, a water-soluble polymer, and a plasticizer; casting the aqueous solution in a suitable substrate; and drying the aqueous solution to provide a thin film. The thin film may be any film as described herein.

In some embodiments, the present disclosure provides a method of making a thin film, comprising providing an aqueous solution comprising PVA, HPMC, xanthan gum, citric acid, tartaric acid, benzoic acid, glycerol, L-lactic acid, and sodium hydroxide; casting the aqueous solution in a suitable substrate; and drying the aqueous solution to provide a thin film.

In some embodiments, the aqueous solution may be cast on a substrate comprising polymer thin sheets, polymer rolls, or foils or rolls made of glass or metal. In some embodiments, the aqueous solution may be cast on a substrate comprising TEFLON® (polytetrafluoroethylene (PTFE)), polyester (PET), polyvinyl chloride (PVC), polyethylene (PE), or polypropylene (PP), or combination thereof. The solution may then be dried to remove the solvent, at temperatures ranging from about 35° C. to about 100° C., for a period of time ranging from about 10 minutes to about 48 hours. The dried film can then be removed from the substrate, for example by peeling, exfoliation, or other mechanical removal. Alternatively, the entire process, including casting and drying, can be carried out in a roll-to-roll process using standard thin film handling equipment. The dried film may be washed to remove any solvents or etchant that may remain. For example, deionized (DI) water can be used to wash the dried film. In certain embodiments, tape casting techniques can be used for casting. The dried film can be cut into smaller pieces or mechanically divided.

In some embodiments, the aqueous solution further comprises an additional buffering agent, as described in detail above. In some embodiments, the additional buffering agent is an organic acid. In some embodiments, the additional buffering agent may include, but is not necessarily limited to, citric acid, tartaric acid, benzoic acid, alginic acid, sodium monocitrate, and any combination thereof.

In some embodiments, the methods described herein may further comprise adding to the aqueous solution a preservative, such as benzoic acid and/or sodium benzoate, as described in detail above.

In some embodiments, the water-soluble polymer may include, but is not necessarily limited to, hydroxypropyl methylcellulose (HPMC), polyvinyl alcohol (PVA), xanthan gum, sodium alginate, carbopol, hydroxyethylcellulose, and any combination thereof. In some embodiments, the water-soluble polymer comprises HPMC. In some embodiments, the water-soluble polymer comprises PVA. In some embodiments, the water-soluble polymer comprises PVA and HPMC. In some embodiments, the water-soluble polymer comprises xanthan gum, PVA and HPMC.

In some embodiments, the PVA has an average molecular weight between about 20,000 and about 200,000.

In some embodiments, the plasticizer may be, but is not necessarily limited to, glycerol, polyethylene glycol, propylene glycol, sorbitol, and any combination thereof. In specific embodiments, the plasticizer is glycerol. In some embodiments, the glycerol is present in the aqueous solution at a concentration between about 0.2% (v/v) and about 4% (v/v).

In some embodiments, the L-lactic acid is present in the aqueous solution at a concentration between about 5 mg/mL and about 20 mg/mL. In some embodiments, the L-lactic acid is present in the aqueous solution at a concentration between about 8 mg/mL and about 15 mg/mL. In some embodiments, the methods comprise using about 8 mg/mL to about 15 mg/mL L-lactic acid.

In some embodiments, the water-soluble polymer is present in the aqueous solution at a concentration between about 1 mg/mL and about 150 mg/mL.

In some embodiments, the methods described herein do not comprise using alginic acid as an additional buffering agent. In some embodiments, the methods described herein do not comprise using alginic acid.

In some embodiments, the PVA has an average molecular weight between about 30,000 and about 186,000, as indicated elsewhere herein. In some embodiments, the methods comprise using about 40 mg/mL to about 80 mg/mL PVA having an average molecular weight of about 20,000 to about 75,000, e.g. about 30,000 to about 70,000. In some embodiments, the methods comprise using about 0 mg/mL to about 10 mg/mL PVA having an average molecular weight of about 75,000 to about 140,000, e.g. about 85,000 to about 124,000. In some embodiments, the methods comprise using about 0 mg/mL to about 8 mg/mL PVA having an average molecular weight of about 140,000 to about 200,000, e.g. about 146,000 to about 186,000. Molecular weight may be any value or subrange within the recited ranges, including endpoints.

In some embodiments, the aqueous solution may comprise about 40 mg/mL to about 80 mg/mL PVA having an average molecular weight of about 20,000 to about 75,000; about 0 mg/mL to about 10 mg/mL PVA having an average molecular weight of about 75,000 to about 140,000; and about 0 mg/mL and about 8 mg/mL PVA having an average molecular weight of about 140,000 to about 200,000. In some embodiments, the aqueous solution may comprise using about 40 mg/mL to about 80 mg/mL PVA having an average molecular weight of about 30,000 to about 70,000 and about 2 mg/mL to about 10 mg/mL PVA having an average molecular weight of about 85,000 to about 124,000. In some embodiments, the aqueous solution may comprise using about 40 mg/mL to about 80 mg/mL PVA having an average molecular weight of about 30,000 to about 70,000 and about 1 mg/mL to about 10 mg/mL PVA having an average molecular weight of about 146,000 to about 186,000. Concentrations or molecular weight may be any value or subrange within the recited ranges, including endpoints.

In some embodiments, the aqueous solution may comprise about 0 mg/mL to about 15 mg/mL HPMC. In some embodiments, the aqueous solution may comprise about 1 mg/mL to about 10 mg/mL HPMC. Concentration may be any value or subrange within the recited ranges, including endpoints. In specific embodiments, the HPMC is HPMC E50. In specific embodiments, the HPMC is HPMC K15.

In some embodiments, the methods comprise using between about 0.1 mg/ml and about 1.5 mg/mL xanthan gum. In some embodiments, the methods comprise using citric acid in a range from about 1 mg/mL to about 8 mg/mL. In some embodiments, the methods comprise using tartaric acid in a range from between about 0.5 mg/mL and about 5 mg/mL. In some embodiments, the methods comprise using benzoic acid in a range from about 0.05 mg/mL to about 0.75 mg/mL. In some embodiments, the methods comprise using about 0.2% (v/v) to about 3% glycerol. In some embodiments, the methods comprise using about 1 mg/mL to about 6 mg/mL sodium hydroxide. Concentrations may be any value or subrange within the recited ranges, including endpoints.

In some embodiments, the thin film has one or more parameters as described elsewhere herein. For example, in some embodiments, the methods described herein produce a thin film where about 2.5 cm² of the thin film has a buffering capacity of at least about 0.2 mEq NaOH. In some embodiments, wherein the thin film produced by the methods described herein has a dispersion time in water of less than about 5 minutes. In some embodiments, the thin film has a dispersion time in water of less than about 4 minutes. In some embodiments, the thin film has a pH after dispersion in saline of less than about 3.7. In some embodiments, the thin films described herein have a pH after dispersion in simulated vaginal fluid (SVF) of less than about 4. In some embodiments, the methods described herein produce a thin film having a bioadhesive strength of about 0.2 to about 1 N. In some embodiments, the methods described herein produce a thin film having a viscosity after dispersion in simulated vaginal fluid (SVF) of at least about 20 cP. In some embodiments, the methods described herein produce a thin film that is stable for at least 2 months at 40° C. and 75% relative humidity. In some embodiments, the methods described herein produce a thin film that is stable for at least 6 months at 40° C. and 75% relative humidity. In some embodiments, the methods described herein produce a thin film having a tensile strength of between about 0.5 Mpa and about 2 Mpa. In some embodiments, the methods described herein produce a thin film having a tensile strength of between about 0.8 Mpa and about 2 Mpa. In some embodiments, the methods described herein produce a thin film having a % elongation of between about 20 and about 50. In some embodiments, the methods described herein produce a thin film having a % elongation of between about 25 and about 40. In some embodiments, the thin films described herein have a toughness of between about 0.06 MJ/m³ and about 0.5 MJ/m³. In some embodiments, the methods described herein produce a thin film having a toughness of between about 0.1 MJ/m³ and 0.35 MJ/m³.

In some embodiments, the aqueous solution is dried at a temperature between 35° C. and 100° C. In some embodiments, the aqueous solution is dried for between 10 minutes and 48 hours.

In some embodiments, the method may further comprise removing the thin film from the substrate.

In embodiments, the solution is mixed in a manner to minimize the incorporation of air into the mixture. In some embodiments, the method may further comprise de-aerating the aqueous solution. De-aerating the solution may be performed using any method. Non-limiting examples include conditioning at room temperature, vacuum treatment or the like, to allow trapped air to escape prior to the drying process. This serves to eliminate bubble and void formation in the final film product, thereby further improving uniformity.

In some embodiments, the method may further comprise cutting the film. In embodiments, the film is cut to a size appropriate for a single dose. In some embodiments, the film is cut to a size between about 1 cm² and about 10 cm². In some embodiments, the film is cut to a size between about 1 cm² and about 8 cm². In some embodiments, the film is cut to a size between about 1 cm² and about 6 cm². In some embodiments, the film is cut to a size between about 1 cm² and about 5 cm². In some embodiments, the film is cut to a size between about 2 cm² and about 10 cm². In some embodiments, the film is cut to a size between about 3 cm² and about 10 cm². In some embodiments, the film is cut to a size between about 2 cm² and about 5 cm². In some embodiments, the film is cut to a size between about 3 cm² and about 5 cm². In some embodiments, the film is cut to a size between about 1 cm² and about 4 cm². In some embodiments, the film is cut to a size between about 2 cm² and about 4 cm². In some embodiments, the film is cut to a size of about 5 cm². Size may be any value or subrange within the recited ranges, including endpoints.

In some embodiments, the casting may comprise pouring, extruding, or printing the aqueous solution onto the substrate.

### Methods of Treatment

In some embodiments, the present disclosure provides a method of contraception, comprising administering any of the thin films described herein, or any of the thin film made by any of the methods described herein. In embodiments, the thin film is administered intravaginally.

In some embodiments, the present disclosure provides a method of preventing transmission of a sexually transmitted infection, comprising administering any of the thin films described herein, or any of the thin film made by any of the methods described herein. In embodiments, the thin film is administered intravaginally.

In some embodiments, the present disclosure provides a method of contraception and preventing a sexually transmitted infection, comprising administering any of the thin films described herein, or any of the thin films made by any of the methods described herein. In embodiments, the thin film is administered intravaginally.

### Kits

In some embodiments, the present disclosure provides a kit comprising a plurality of containers, each container comprising a thin film as described in detail above, or any of the thin films made by any of the methods described above.

Non-limiting examples of suitable containers include a bottle, tube, or box. In embodiments, the container is a pouch or package. In embodiments, the pouch or package is made of paper, plastic, and/or or metal. In embodiments, the pouch or package is lined with paper, plastic, and/or or metal. In embodiments, the pouch or package comprises aluminum foil. In embodiments, the pouch or package includes a high permeability barrier.

In some embodiments, the kit may further comprise instructions for use of the thin film as a contraceptive.

In some embodiments, the may further comprise an applicator. In some embodiments, the applicator is configured to insert the thin film into a vaginal cavity.

### EXAMPLES

One skilled in the art would understand that descriptions of making and using the films described herein is for the sole purpose of illustration, and that the present disclosure is not limited by this illustration.

### Example 1. Preparation of Thin Film

Previous formulations include a vaginal gel (referred to herein as “gel composition”) that serves to act as a contraceptive. The buffering capacity was defined as the amount of sodium hydroxide required to raise the pH of the gel from 3.5 to 5. The buffering capacity of the vaginal gel was found to be 0.34 meq.

Prior to development of a thin film formulation, polymer-polymer compatibility was investigated using Fourier Transform Infrared (FTIR). The FTIR is commonly used to investigate the chemical incompatibility between drugs and excipients. Pure HPMC, Xanthan, and the mixture of polymers were investigated for compatibility using the ATR-FTIR Perkin Elmer spectrum 400 USA, FTIR instrument. The spectra were obtained at a frequency range of 4000-500 cm⁻¹ with a resolution of 4 cm⁻¹ and 32 scanning rates.

Pure alginic acid, Xanthan gum, and the mixture of both polymers were investigated for compatibility using the ATR-FTIR Perkin Elmer spectrum 400 USA, FTIR instrument. The spectra were obtained at a frequency range of 4000-500 cm⁻¹ with a resolution of 4 cm⁻¹ and 32 scanning rates. The powders were placed on the ATR diamond crystal and subsequently, a force was applied with the use of the clamp to ensure that the samples made adequate contact with the diamond crystal.

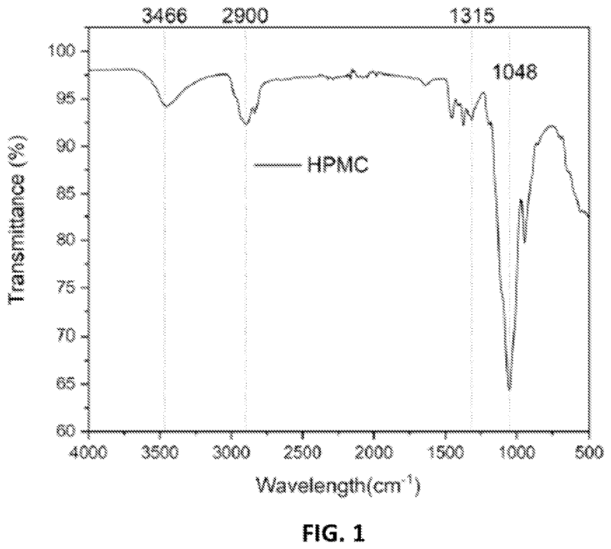

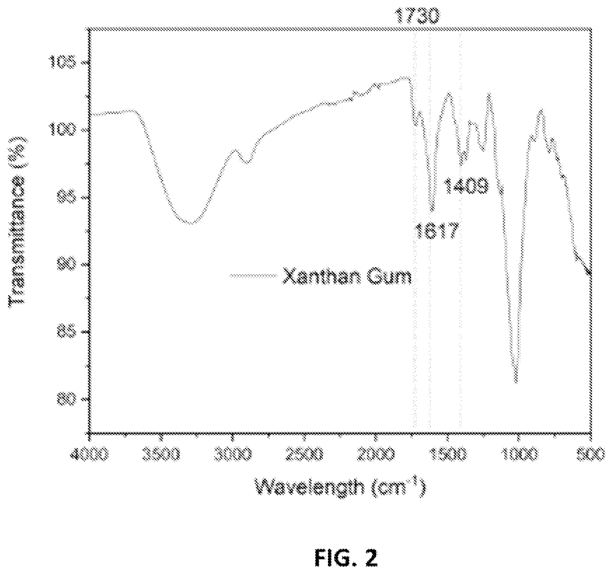

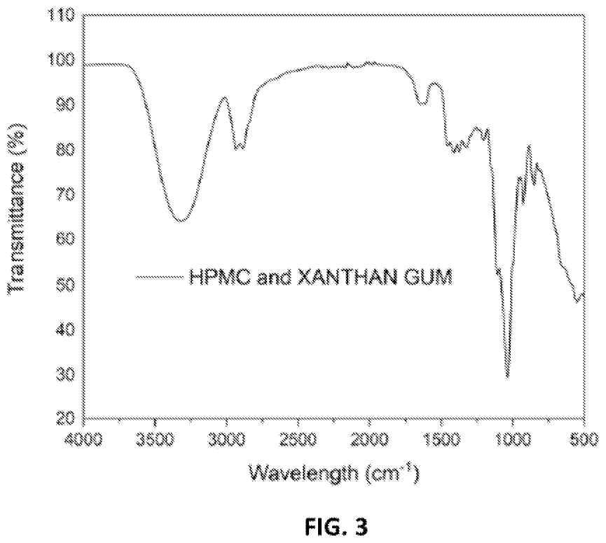

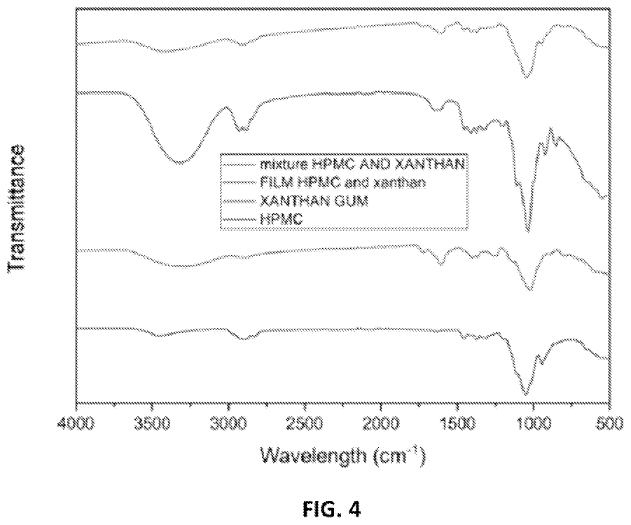

FTIR studies conducted on pure polymers and mixture are shown in FIGS. 1-4. The use of FTIR to evaluate the drug-excipients or excipient-excipient interactions is widely reported in the literature. This evaluation is normally based on known and characteristic stretching regions of the FTIR spectra that may disappear or have reduced intensity due to electrostatic interactions and/or hydrogen bonds between the components. From the spectra of HPMC, there was a strong band at 3466 and 2900 cm⁻¹ due to OH and alkyl C—H group respectively. Furthermore, there is also a band at 1315 cm⁻¹ which can be attributed to the stretching of C—O-ether linkage (Soni et al. Int Curr Pharm J 6:61 (2018)). As reported in the literature, the spectra of Xanthan gum show a strong band at 1730, 1617, and 1409 cm⁻¹ which can be ascribed to the C—O stretch, asymmetric and symmetric vibrations of COO—in pyruvate and glucuronate group (Lal et al. Mat Science Eng 79 (2017)). From the result obtained there was no remarkable change in the FTIR Spectra of the mixture, meaning that the results revealed no physical or chemical interactions. No change in the peaks of the polymers even after forming a film. In summary, the result clearly shows that there was no interaction between the blend of polymers used in film fabrication.

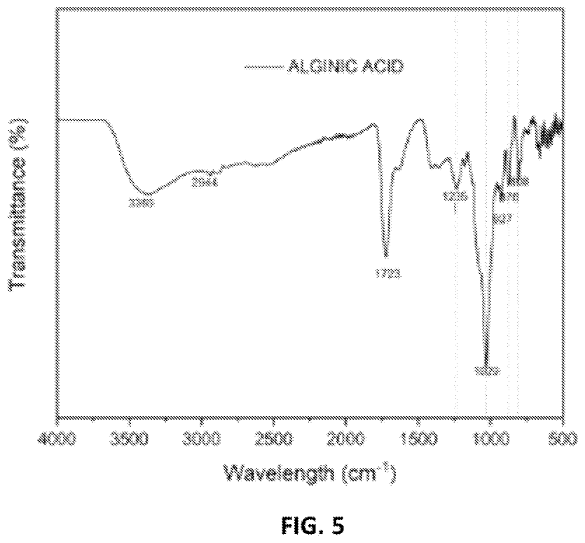

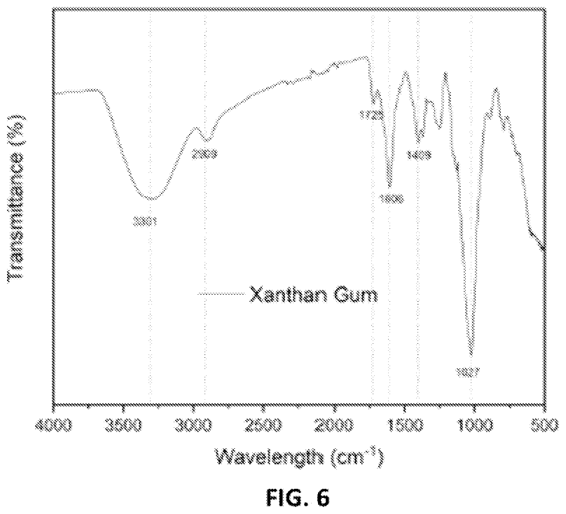

The FTIR spectra of alginic acid, Xanthan gum, and the mixture are shown in FIGS. 5, 6, and 7, respectively. As shown in FIG. 5, the spectrum of alginic acid is consistent with those reported in the literature (Wang et al. Nanomaterials 8:76 (2018); Rigo et al. Int J Pharm 322 (1): 36-43 (2006)). The alginic acid FTIR spectrum displays a very broad peak/band at 3365 cm⁻¹ which can be ascribed to the O—H group. The other major peaks in Alginic acid are the band at 2944, 1723 1029 cm⁻¹ which were assigned to C—H, C═O, and C—O group, respectively. Also, the polysaccharide display peaks at 1235, 1168 cm⁻¹. In the fingerprint region, the peak at 941 and 927 cm⁻¹, can be ascribed to the C—O stretching from the pyranose ring while the peak at 876 and 808 cm⁻¹ belong to the guluronic and the mannuronic acid block respectively.

Xanthan Gum FTIR spectra are characterized by a broad spectrum at 3404 cm 1 which is a typical stretch for OH group, then another peak at 2909 which can be attributed to the C—H group stretching. There is a major absorption band at 1735.67 cm⁻¹, 1606 cm⁻¹ and1409.5 cm⁻¹ due to C—O stretch, asymmetric and symmetric vibrations of COO—in pyruvate and glucuronate group (Lal et al. Mat Science Eng 79 (2017)).

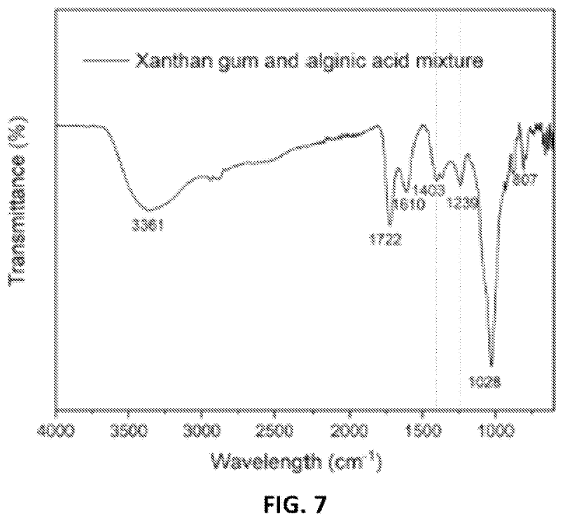

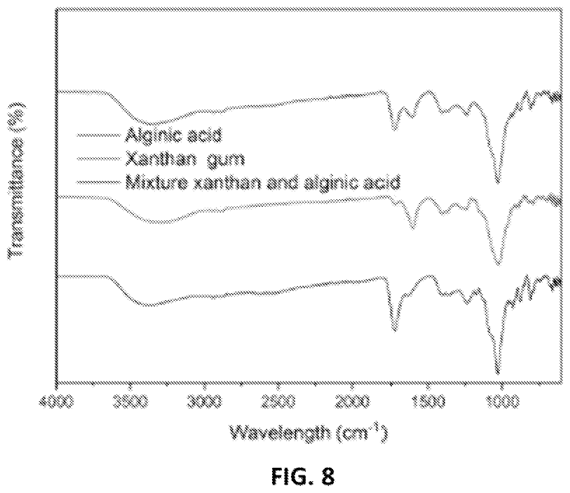

From the physical mixing, the combination shows a peak at 3361, 1722, 1610, 1403, 1239, 1028, and 807 cm⁻¹ (FIGS. 7 and 8). The result clearly shows that there is no significant band shift when both polymer blends are combined. The FTIR spectrum of the physical mixture of alginic acid and Xanthan gum showed that there is no significant chemical interaction between the two polymers. Furthermore, all the functional peaks in alginic acid and Xanthan gum are still present in the spectra of the mixture. All the characteristic peaks of alginic acid and Xanthan gum still appear in the spectra of the mixture. Also, there were no new bands observed in the polymer-polymer blends, which confirms that no new chemical bonds were formed between the drug and polymers studied (FIGS. 7 and 8).

These preliminary results showed the possibility of using a blend of HPMC and Xanthan gum for the fabrication of a vaginal film. The result confirmed that there is no significant band shift or appearance of a new peak when both polymer blends are combined.

### Blank Film Fabrication—Trial 1

Based on the literature review, HPMC as film-forming polymer and glycerin as plasticizer, were used in a preliminary trial for film fabrication. Different concentrations of HPMC were considered. Solvent casting method was used in film fabrication. The HPMC E50 was added to water that was already heated to 60° C., the mixture was stirred at 1000 rpm and allowed to cool to room temperature while still stirring at 1000 rpm. Glycerol was added to the polymer solution and this was allowed to mix thoroughly until a clear homogenous solution was formed. The smooth solution was poured into a glass Petri plate and this was dried at 60° C. for 24 hours. The dried film was then carefully removed from the glass plate and cut into pieces.

These experiments were conducted to get started on the film preparation. The use of glycerin 2% and HPMC E50 (1.5%) results in a thin film. Batch F1 formed film with powder on the edge of the film, F2 and F5 show some air bubbles. F5 produced a very thick film with some air bubbles. F6 formed thin film.

### Blank Film Fabrication—Trial 2

Blank film was fabricated using different grades of HPMC (E3, E5, E15, and E50) and a blend of polymers (HPMC and Xanthan Gum). HPMC was considered for the fabrication of blank film and using glycerin as a plasticizer. However, HPMC alone despite forming a good film results in a film with poor mechanical strength. As a result, a blend of HPMC and Xanthan gum was also considered for film development. The addition of Xanthan gum was expected to increase the bioadhesive strength of vaginal films and formulation. The following flowchart describes the method of making the film:

The final solution was poured into a glass Petri plate and dried at 50° C. for 24 hours. The dried film was then carefully removed from the glass plate and cut into pieces. Table 1 below shows the formulation compositions of various tested compositions.

TABLE 1
Formulation Composition

| Components | F7 | F8 | F9 | F10 | F11 | F12 | F13 |
|---|---|---|---|---|---|---|---|
| HPMC E3 | 2.5 | | | | | | |
| HPMC E5 | | 2.5 | | | | | |
| HPMC E15 | | | 2 | | | | |
| HPMC E50 | | | | 4 | | | |
| HPMC E50 | | | | | 7 | | |
| HPMC E50 | | | | | | 3 | 3 |
| Xanthan | | | | 0.4 | | 0.2 | |
| gum | | | | | | | |
| Glycerin | 2.5 | 2.5 | 2.5 | 2.5 | 2.5 | 2.5 | 2.5 |
| Citric acid | | | | | | | |
| Water qs | 100 | 100 | 100 | 100 | 100 | 100 | 100 |

The thickness of the fabricated films was measured at five different locations (center and at four different spots) using calibrated digital Vernier caliper (Mitutoyo, Japan). The fabricated film was characterized for appearance, color, flexibility, peelability, and thickness, as shown in Table 2.

TABLE 2

| Tests | F7 | F8 | F9 | F10 | F11 | F12 | F13 |
|---|---|---|---|---|---|---|---|
| Appearance | White | White | White | White | White | White | White |
| & color | transparent | transparent | transparent | transparent | transparent | transparent | transparent |
| | film | film | film | film | film | film | film |
| Pealability | Difficult to | Difficult to | Thin film, | Polymers | Too thick | Good film | Good film |
| | peel | peel | very | precipitate | did not | | |
| | | | fragile | at the edge | spread | | |
| | | | | of the plate | easily on | | |
| | | | | | Teflon | | |
| Others | Poor film | Poor | Fragile | | Very hard | | |
| | need more | mechanical | | | film | | |
| | polymers | properties | | | | | |
| Average | — | 0.05 | 0.05 | — | 0.21 | 0.08 | 0.06 |
| thickness | | | | | | | |
| (mm) | | | | | | | |

### Blank Film Fabrication—Trial 3

The purpose of the present investigation was to explore the possibility of using a combination of polymers such as HPMC, Xanthan gum, sodium alginate, PVA, and carbopol to formulate blank and acid buffering film for vaginal film fabrication. The acid buffering film formulated was titrated against sodium hydroxide to check the acid buffering capacity. A variety of polymers are available for use in formulating vaginal films. The selection of polymer is an important parameter for the successful development of the film. Individual polymers and combinations of the polymers can be used to obtain the derived film characteristic and properties.

The blank films were prepared by the solvent casting method. Briefly, aqueous solution was prepared by dissolving HPMC (was added to water that was already heated to 60° C.) and PVA (added to water already heated to temperature 90° C.), xanthan gum and sodium alginate in water. The mixture was stirred at 1000 rpm and allowed to cool to room temperature while still stirring at 1000 rpm. This was followed by the addition of glycerol for achieving a plasticizing effect. The resulting viscous solution was allowed to mix thoroughly until a clear homogenous solution was formed. Before casting, it was important to ensure that there were no air bubbles in the solution. The mixture was allowed to stir for about 4 hours and the final solution was kept for 1 hour to remove any entrapped air bubbles. The resulting viscous solution was poured into the PFA Teflon petri dish and then dried in an oven at 40° C. for 24 h (see Table 3). The dried films were carefully removed from the Teflon plate and checked for any imperfections or air bubbles and stored in aluminum foil until needed for use. The films were carefully cut into the required size.

TABLE 3
Composition of the film

| | | Xanthan | | Sodium | | | | |
|---|---|---|---|---|---|---|---|---|
| | HPMC | gum | PVA | alginate | Carbopol | Glycerol | Water | |
| Batch | (mg) | (mg) | (mg) | (mg) | (mg) | (mL) | (QS) | Comments |
| F1 | 600 | 200 | | | | 1 | 50 | Film formed (solution a |
| | | | | | | | | bit viscous) |
| F2 | 600 | 100 | | | | 1 | 50 | Film formed very thin |
| | | | | | | | | and smooth |
| F3 | 600 | | 400 | | | 1 | 50 | Film formed |
| F4 | 600 | | 200 | | | 1 | 50 | Thin-film very smooth |
| F5 | 600 | | | 600 | | 1 | 50 | Yellowish colour film |
| | | | | | | | | due to sodium alginate |
| F6 | 600 | | | 300 | | 1 | 50 | Slightly yellowish colour |
| F7 | 600 | | | | 1000 | 1 | 50 | Poor - Couldn't peel and |
| | | | | | | | | didn't dissolve very well |
| F8 | 600 | | | | 500 | 1 | 50 | Poor - Couldn't peel and |
| | | | | | | | | didn't dissolve very well |
| F9 | 600 | 160 | 200 | | | 1.5 | 50 | Film formed |
| F10 | 600 | 160 | 100 | | | 1.5 | 50 | Film formed |
| F11 | 600 | 100 | 100 | | | 1.5 | 50 | Film formed |
| F12 | 600 | 100 | | | | 1.5 | 50 | Film formed |
| F13 | 600 | | 100 | 400 | | 1.5 | 50 | Film formed yellowish |
| | | | | | | | | colour film |
| F14 | 600 | | 100 | 300 | | 1.5 | 50 | Film formed yellowish |
| | | | | | | | | colour film |
| F15 | 600 | 160 | 100 | | | 1.5 | 50 | Film formed |
| F17 | 600 | 160 | 100 | | | 2.0 | 50 | Sticky due to excess |
| | | | | | | | | glycerol |

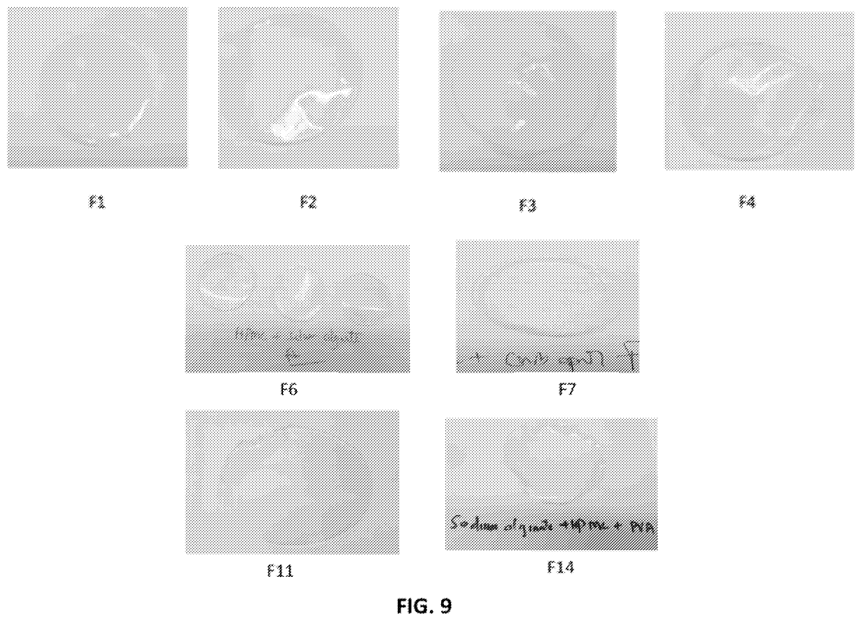

The film-forming capacity of the polymer combination and visual appearance are shown in FIG. 9 and Table 4. This aspect of the experiment shows the possibility of forming a vaginal film by using a combination of polymers. All the combinations show good film-forming capability with glycerol as the plasticizer.

TABLE 4
Results of exploring different polymers for film development

| | Combination | Film-forming | Visual |
|---|---|---|---|
| s/n | of polymers | capability | appearance |
| 1 | HPMC and Sodium alginate | Good | Smooth and light yellowish |
| 2 | HPMC and PVA | Good | Smooth and transparent |
| 3 | HPMC and Xanthan gum | Good | Smooth and transparent |
| 4 | HPMC and Carbopol | poor | Poor (need to improve) |
| 5 | HPMC, PVA, and Xanthan gum | Good | Smooth and transparent |
| 6 | HPMC, PVA, and sodium alginate | Good | Smooth and slightly light yellowish |

This experiment provides the groundwork for the next stage of the experiment which is loading of the acid buffering agent into the films. Glycerol ratio and the concentration of the polymer was varied to obtain the derived film characteristic and properties.

### Fabrication of Acid Buffering Film

This aspect involves the loading of the acid buffering ingredient into the polymer solution. The preparation of the acid buffering film consists of 3 different processes. In the first stage dispersion of the acid buffering ingredients was weighed and dissolved in a small amount of water (5 mL). Then alginic acid was also weighed and added to water (15 mL). These two mixtures were added together to form a homogenous mixture. This was then added to the polymer solution containing both polymers and glycerol. The final mixture was allowed to stirred at 500 RPM for about 4 hours to ensure proper mixing. Care was taken to prevent air entrapment. The final pH of the solution was recorded before casting into the PFA Teflon petri dish and then dried in an oven at 40° C. for 24 h. Table 5 shows the acid buffering film composition.

TABLE 5
Formulations of films F20-F23

| Ingredients | F20 | F21 | F22 | F23 |
|---|---|---|---|---|
| HPMC (mg) | 600 | 600 | 600 | 600 |
| PVA (mg) | 100 | 100 | | |
| Sodium | 300 | | 300 | |
| alginate (mg) | | | | |
| Xanthan gum | | 160 | | 160 |
| (mg) | | | | |
| Glycerol (ml) | 1.52 | 1.52 | | |
| Citric acid (mg) | 160 | 160 | | |
| Lactic acid | 340 | 340 | 340 | 340 |
| (mg) | | | | |
| Tartaric acid | 72 | 72 | 72 | 72 |
| (mg) | | | | |
| Sodium | 240 | 240 | 240 | 240 |
| hydroxide (mg) | | | | |
| Benzoic acid | | | | |
| (mg) | | | | |
| Alginic acid | 1360 | 1360 | 1360 | 1360 |
| Water (QS) to | 50 | 50 | 50 | 50 |
| mL | | | | |

This preliminary work shows the possibility of formulating acid buffering vaginal film with a combination of polymers. This work will now progress to the optimization of the desired buffering capacity, acceptable dimension, and mechanical properties. The acid buffering ability of this film was evaluated to find out the amount of sodium hydroxide required to raise the pH of 2.5×2.5 cm of the film from pH 3.5 to 5.

### Example 2. Determination of the Acid Buffering Capacity of the Film

This study investigates the acid buffering capacity of the vaginal film. The acid buffering ability of this film was evaluated to find out the amount of sodium hydroxide required to raise the pH of 2.5×2.5 cm of the film from pH 3.5 to 5. Briefly, 1 M standardized sodium hydroxide was added at 10-20 μL increments to the film which was previously dispersed in 10 mL of normal saline under stirring. NaOH solution was added in 10 μl increments under constant stirring. The pH was measured after each addition. Stirring was stopped, while pH is measured. The titration curves were used for determining the relevant buffering capacity, defined previously as the amount of sodium hydroxide required to reach a pH value of 5.

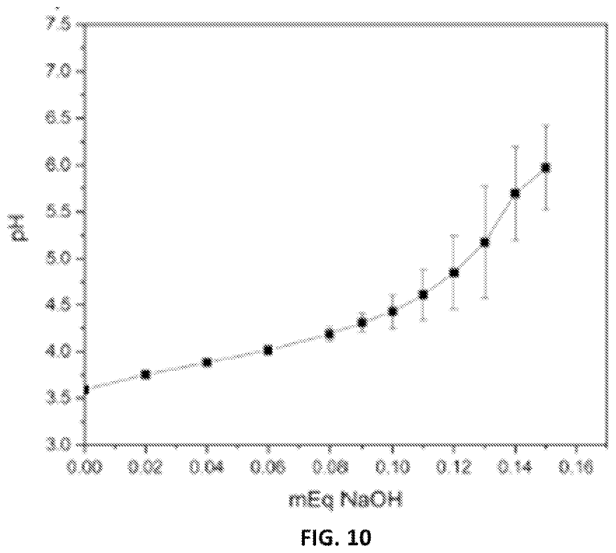

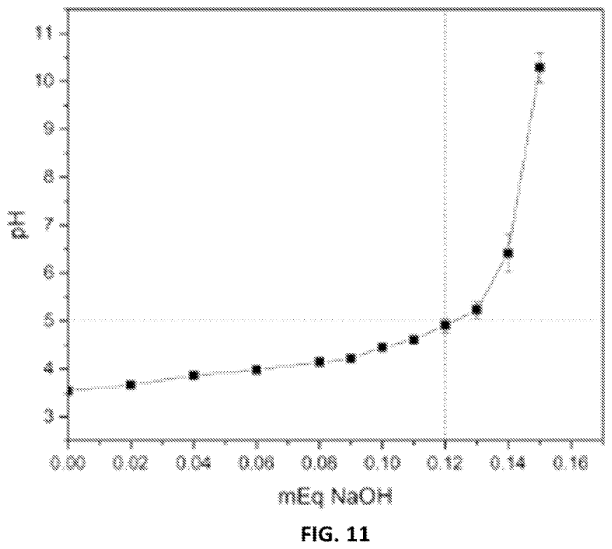

The acid buffering capacity of the film after titration against 1N sodium hydroxide was determined using a pH meter. FIGS. 10 and 11 show the pH titration profile of the film against NaOH. The buffering capacity of the film is defined as the amount of sodium hydroxide that is required to raise the pH of the film from 3.5 to 5. From the result, the amount required is 0.12 meq of sodium hydroxide for HPMC-PVA-Xanthan gum film (F21) and 0.125 meq of sodium hydroxide for HPMC-PVA-sodium alginate film (F20). Film 22 and 23 did not give any good acid buffering effect at this stage. This needs to be improved in the next stage of the experiment.

The preliminary buffering activity of the fabricated film was found to be 0.12 mequivalents which is less than 0.32 mequivalents for the gels. This requires further optimization of the acid buffering properties of the film.

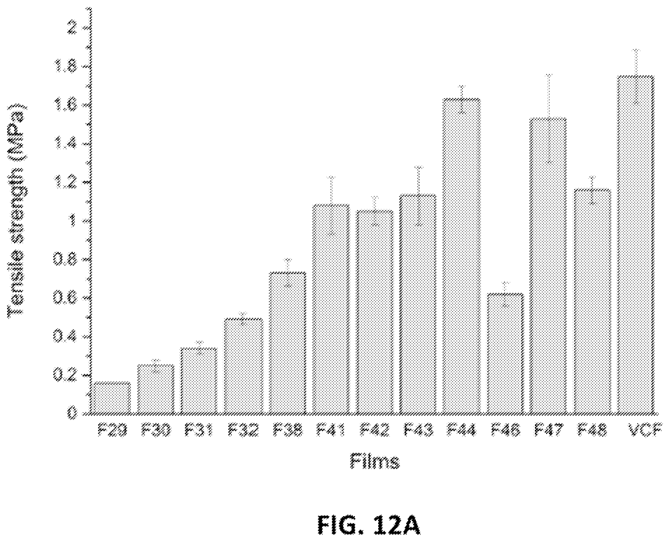

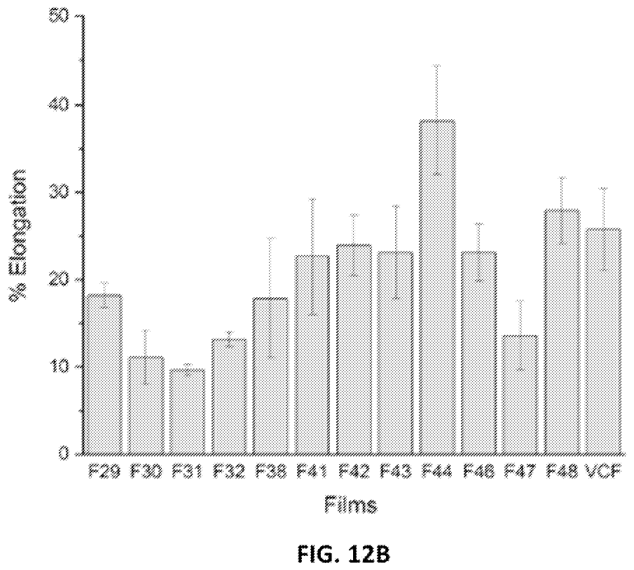

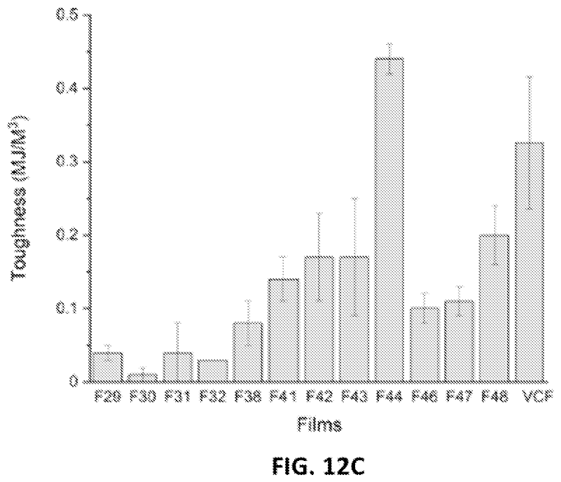

Various formulations were tested (Table 6). An ideal vaginal film is expected to possess moderate tensile strength, high elongation ability, and low elastic modulus. Table 7 and FIGS. 12A-12C show results obtained from various tested formulations.

TABLE 6
Fabricated films and compositions

| | | PVA | | | | | | | | |
|---|---|---|---|---|---|---|---|---|---|---|
| | PVA | 30,0000- | PVA | | | | | Acid | | |
| | 15,0000 | 70,000 | 100,0000 | HPMC | | Xanthan | | buffering | | |
| Film | (HD: 86-89) | (HD: 86-89) | (HD: 86-89) | E50 | HPMC | gum | | agents | Water | |
| Sample | (mg) | (mg) | (mg) | (mg) | K15(mg) | (mg) | Glycerol | (mg) | (QS) | Comments |
| F29 | 1500 | | | 1000 | | 200 | 1.2 | ✓ gel | 50 | Low TS |
| | | | | | | | | composition | | (TS- |
| | | | | | | | | | | tensile |
| | | | | | | | | | | strength) |
| F30 | 2000 | | | 1000 | | 200 | 1.2 | ✓ gel | 50 | Low TS |
| | | | | | | | | composition | | |
| F31 | 2500 | | | 1500 | | 200 | 1.2 | ✓ gel | 50 | Low TS |
| | | | | | | | | composition | | |
| F32 | 3500 | | | 1000 | | 200 | 1.2 | ✓ gel | 50 | Low TS |
| | | | | | | | | composition | | |
| F33 | | 2000 | | | 250 | 100 | 0.3 | | 50 | Poor |
| | | | | | | | | | | peelability |
| F34 | | 3000 | | | 250 | 100 | 0.4 | | 50 | Poor |
| | | | | | | | | | | peelability |
| F35 | | 3000 | | | 375 | 150 | 1.00 | | 50 | Imperfection - |
| | | | | | | | | | | air |
| | | | | | | | | | | bubble |
| | | | | | | | | | | and |
| | | | | | | | | | | uneven |
| | | | | | | | | | | surface |
| F36 | | 1250 | | | 250 | 150 | 1.50 | | 50 | sticky |
| F37 | | 1500 | | | 375 | 200 | 1.50 | | 50 | sticky |
| F38 | | 1500 | | | 750 | 200 | 0.85 | | 50 | Good film |
| F39 | 500 | 2000 | 500 | | 250 | 200 | 0.85 | | 50 | Too |
| | | | | | | | | | | viscous |
| | | | | | | | | | | and |
| | | | | | | | | | | difficult to |
| | | | | | | | | | | form |
| | | | | | | | | | | uniform |
| | | | | | | | | | | film |
| F40 | 1000 | 1000 | 1000 | | 200 | 50 | 0.85 | | 50 | Difficult |
| | | | | | | | | | | to remove |
| | | | | | | | | | | from |
| | | | | | | | | | | casting |
| | | | | | | | | | | surface - |
| | | | | | | | | | | poor |
| | | | | | | | | | | peelability |
| F41 | 700 | 2500 | | | 250 | 50 | 0.8 | | 50 | Good film |
| F42 | | 3000 | | | 250 | 40 | 0.7 | | | Good film |
| F43 | | 2900 | 100 | | 250 | 50 | 0.8 | | 50 | Good film |
| F44 | | 3000 | 200 | | 250 | 40 | 0.9 | | 50 | Good film |
| F45 | | 3000 | | | 250 | 40 | 0.9 | ✓ gel | 50 | Poor film |
| | | | | | | | | composition | | with slight |
| | | | | | | | | | | alginic |
| | | | | | | | | | | particles |
| | | | | | | | | | | on the |
| | | | | | | | | | | surface of |
| | | | | | | | | | | the film |
| F46 | | 3000 | | | 250 | 40 | 0.9 | ✓ gel | 60 | Acceptable |
| | | | | | | | | composition | | |
| F47 | | 3000 | | | 250 | 40 | 0.8 | ✓ New | 50 | Good but |
| | | | | | | | | composition | | slightly |
| | | | | | | | | without | | dry |
| | | | | | | | | alginic | | |
| | | | | | | | | acid | | |
| F48 | | 3000 | | | 250 | 40 | 0.9 | ✓ new | 50 | Good |
| | | | | | | | | composition | | |
| | | | | | | | | without | | |
| | | | | | | | | alginic | | |
| | | | | | | | | acid | | |

TABLE 7
Tensile strength results

| | | % | Toughness |
|---|---|---|---|
| Films | TS (Mpa) | Elongation | (MJ/m³) |
| F29 | 0.16 ± 0.01 | 18.17 ± 1.36 | 0.04 ± 0.02 |
| F30 | 0.25 ± 0.04 | 11.09 ± 3.13 | 0.01 ± 0.02 |
| F31 | 0.34 ± 0.04 | 9.62 ± 0.70 | 0.04 ± 0.06 |
| F32 | 0.49 ± 0.04 | 13.14 ± 0.84 | 0.03 ± 0.01 |
| F38 | 0.73 ± 0.08 | 17.80 ± 6.39 | 0.08 ± 0.04 |
| F41 | 1.08 ± 0.16 | 22.64 ± 6.68 | 0.14 ± 0.04 |
| F42 | 1.05 ± 0.08 | 23.92 ± 3.43 | 0.17 ± 0.03 |
| F43 | 1.13 ± 0.07 | 23.13 ± 5.36 | 0.17 ± 0.06 |
| F44 | 1.63 ± 0.20 | 38.23 ± 6.13 | 0.44 ± 0.09 |
| F46 with Alginic acid | 0.62 ± 0.02 | 23.11 ± 3.33 | 0.10 ± 0.03 |
| F47 without alginic, | 1.53 ± 0.24 | 13.59 ± 3.92 | 0.108 ± 0.03 |
| less glycerol and | | | |
| drying at higher temp | | | |
| F48 without alginic | 1.16 ± 0.08 | 27.91 ± 3.82 | 0.159 ± 0.05 |

TABLE 8
Acid buffering ingredients and composition

| | | Gel | New |
|---|---|---|---|
| | | composition | composition |
| | Function/ | Amount/ | Amount/ |
| Ingredients | purpose | Weight (mg) | Weight (mg) |
| Citric acid (mg) | Acid | 250 | 200 |
| Lactic acid (μL) | Acid | 440 | 675 |
| Tartaric acid (mg) | Acid | 112.5 | 125 |
| Benzoic acid (mg) | Preservative | 15.5 | 12.5 |
| Sodium hydroxide (mg) | pH adjustment | 375 | 175 |
| Alginic acid | Thickening and | 1700 | 0 |
| | suspending agent | | |
| | removed | | |
| Final pH | | 3.4 | 3.16 |

Alginic acid was removed from the final formulation because as the concentration of the PVA was increased, the alginic acid was less soluble, leaving yellowish particles on the surface of the film. Despite the benefits (good water swelling ability and increasing viscosity capability) of alginic acid, it was affecting the aesthetic appearance of the film as well as the mechanical strength. It worked well in the gel composition, but not in the film. The initial plan was to tap into the acidic nature for acid buffering purposes and for improving viscosity. Secondly, it may be useful if there is a calcium-containing ingredient in the formulation that can aid in crosslinking, which may likely translate to improved TS. For the purposes of the present disclosure, the use of higher concentration of PVA will increase the TS and viscosity. Increase in PVA resulted in films with higher TS as evident in film F41-F44, F47, and F48 (see FIG. 12A). PVA with higher molecular weight resulted in increased TS while higher glycerol concentration improved elongation (FIG. 12B).

With the new buffering composition (see Table 8), a series of adjustments were done until a final casting pH solution of 3.41 was obtained. The acid component with pH of 3.16 was added to the film forming polymers blend (initial pH 5.75) and the final pH before casting was 3.41.

It was observed that the TS of the acid loaded film with alginic acid component was reduced when compared to the blank from 1.69 MPa to 0.63 MPa. However, from using acid buffering component without alginic acid, TS of 1.16 MPa was obtained. In addition, by manipulating the glycerol ratio TS similar to vaginal contraceptive film (VCF) was obtained. Film F47 without alginic acid and less glycerol felt a bit hard. F48 possessed good TS and elongation. The objective was to have a film with desirable TS which is not too stiff and hard.

In conclusion, PVA with higher molecular weight improved tensile strength. Higher concentrations of glycerol as plasticizer improved elongation of the film. Acid buffering components with alginic acid result in reduced TS, while acid buffering components without alginic acid show minimal impact on TS. Combinations of different grades may allow for controlled release of the acid components from the film. Characterization of film F48 was continued for acid buffering capacity.

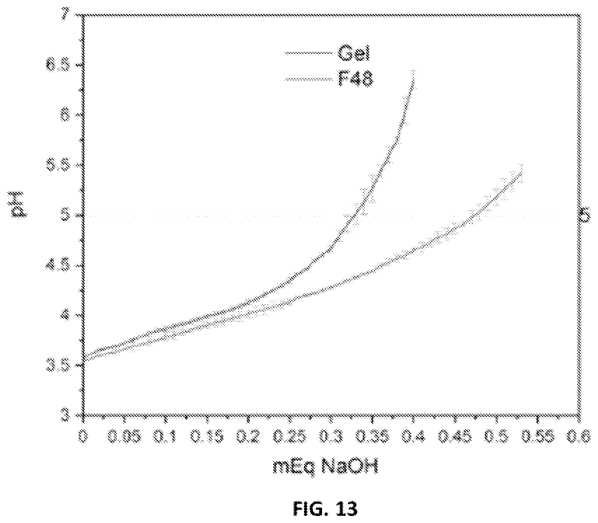

FIG. 13 shows the buffering capacity of vaginal film F48 against 1N sodium hydroxide (size 5×5 cm and thickness 0.19±0.02).

As seen in FIG. 13, Film F48 possesses better acid buffering capacity than the gel composition. This increase in buffering capacity can be attributed to the fact that the new acid buffering component contains more lactic acid (an increase from 440 mg to 675 mg) and tartaric acid (112.5 to 125 mg). The acid composition also contains less sodium hydroxide. The buffering capacity of the film was 0.47 mEq compared with 0.33 mEq for the gel.

### Example 3: Vaginal Films Optimized for Mechanical Properties

To improve the TS of the Film 48, three variables were considered for manipulation: 1) Increase concentration of PVA and use higher Mw PVA with good water solubility and degree of hydrolysis in the range of 86-89 (PVA Mw 85,000-124,000 and 146,000-186,000); 2) Increase concentration and different grades of HPMC (HPMC E50 and HPMC K15); and 3) Vary the ratio of glycerol to control elongation and TS.

For the improvement of the TS, four grades of PVA with a degree of hydrolysation (86-98%) and Mw were utilized in the fabrication of the film. Two additional grades of PVA were tested (PVA Mw 85,000-124,000 and 146,000-186,000).

Hydroxypropyl methylcellulose (HPMC) possesses excellent film forming property and is commercially available in several grades. They contain varying degrees of methoxyl and hydroxypropoxyl substitution (Gustafsson et al. Eur J Pharm Sci 9:171-184 (2000) as well as different viscosity range from 4000-100,000 mPas. HPMC grade, concentration and Mw can also alter/influence film mechanical properties. In order to improve the TS of the film, different ratios of HPMC and PVA were investigated or considered. Plasticizers such as glycerol are commonly incorporated with polymeric film formers to improve film flexibility. Glycerol is commonly used to plasticize PVA/HPMC blend. By manipulating the amount of glycerol used in the film fabrication we can also control the elongation as well as maintain a relatively good TS.

The aim of this study was to investigate the effects of using varying concentration and grade of PVA, HPMC as well as ratio of plasticizer (glycerol) on the mechanical properties of the film with a clear focus to use this outcome to improve the TS of the vaginal film.

Materials used included HPMC E50, HPMC K15, PVA (different grades with MW 15,000, 30,000-70,000, 100,000, 85,000-124,000 and 146,000-186,000), and Xanthan gum. The acid buffering components included citric acid, tartaric acid, sodium hydroxide, benzoic acid, alginic acid. Texture Analyzer (TA.X.plus Texture Analyzer, Stable Micro Systems, UK), petri dish, stirrer with heating system.

Table 9 shows the composition of the ingredients/polymers used in the fabrication of each film. HPMC was weighed and soaked for about 30 min while PVA was heated to 80° C. to obtain a clear solution. The films were casted on petri dishes and allowed to dry at 45° C. for 24 h. They were further peeled and cut into 3.5×2.2 cm² area for the mechanical test. The films were screened and evaluated for imperfection, surface roughness and peelability without rupturing. Film without imperfection were further evaluated for mechanical test.

TABLE 9
Thin Film Formulations F48 to F71

| | PVA | PVA | PVA | PVA | | | | | | | |
|---|---|---|---|---|---|---|---|---|---|---|---|
| | Mw | 30,000- | 85,000- | 146,000- | | | Xanthan | | | | |
| | 15,000 | 70,000 | 124,000 | 186,000 | HPMC | HPMC | Gum | | Acid | Water | |
| Film | (mg) | (mg) | (mg) | (mg) | E50(mg) | K15(mg) | (mg) | Glycerol | buffering | (QS) | Comments |
| F48 | | 3000 | | | | 250 | 40 | 0.9 | √ | 50 | good |
| F49 | | 3500 | | | | 750 | 40 | .1 | √ | 50 | Too thick |
| | | | | | | | | | | | solution |
| | | | | | | | | | | | Air |
| | | | | | | | | | | | bubbles |
| F50 | | 2500 | | | | 1000 | 40 | 1 | √ | 50 | Air |
| | | | | | | | | | | | bubbles |
| F51 | | 4500 | | | | 250 | 40 | 1 | √ | 50 | |
| F52 | | 4000 | | | | 250 | 40 | 1 | √ | 50 | |
| F53 | | 5000 | | | | 250 | 50 | 1 | √ | 50 | |
| F54 | | 2000 | 2000 | | | 250 | 50 | 1 | √ | 50 | |
| F55 | 1000 | | 2000 | | | 250 | 50 | 1 | √ | 50 | |
| F56 | | 3000 | | | | 250 | 40 | 1 | √ | 50 | |
| F57 | | 5000 | 100 | | | 250 | 50 | 1 | √ | 50 | |
| F58 | | 6000 | | | | 250 | 50 | 1.3 | √ | 50 | |
| F59 | | 1000 | 2000 | | | 250 | 50 | 1.5 | √ | 50 | |
| F60 | 1000 | 7000 | | | | 250 | 50 | 1 | √ | 50 | |
| F61 | 1500 | | 2000 | | | 250 | 50 | 1.2 | √ | 50 | |
| F62 | | | 1500 | | | 250 | 50 | 1.2 | √ | 50 | |
| F63 | | 500 | 2000 | 500 | | 250 | 50 | 1.2 | √ | 50 | |
| F64 | | 2000 | 700 | 300 | | 250 | 50 | 0.9 | √ | 50 | |
| F65 | | 750 | 2000 | 250 | | 1000 | 50 | 0.9 | √ | 50 | |
| F66 | | 600 | 2000 | 400 | | 500 | 50 | 1.0 | √ | 50 | |
| F67 | | | 4000 | 200 | 500 | | 50 | 0.9 | √ | 50 | |
| F68 | | | 500 | | 2000 | | 50 | 1.0 | √ | 50 | |
| F69 | | 1500 | | | 1500 | | 50 | 1.0 | √ | 50 | |
| F70 | | 3000 | 400 | | 250 | | 40 | 0.925 | √ | 50 | |
| F71 | | 3000 | | 300 | 250 | | 40 | 0.95 | √ | 50 | |

The mechanical properties of the films were determined using a Texture Analyzer (TA.XT.plus Texture Analyzer, Stable Micro Systems, U.K.) as previously described by Garg et al (Pharm Res 22 (4): 584-595 (2005)), but slight changes to the size of the film tested. Briefly, the strip of blank films and acid-buffering films cut into 2.2×3.5 cm rectangles were placed between the tensile grip and was tightened to the instrument with the use of the clamp. The lower clamp was fixed during the measurement and the film was pulled by the upper clamp at a rate of 1.00 mm/s. The gap between the lower and upper clamp were kept at a distance of 150 mm (thickness of 0.20 mm was used for the mechanical testing). From the instrument software, the tensile strength and elongation at break were determined. For this experiment, three or more measurements were made for the samples tested.

Results showed that molecular weight, concentration, a grade of PVA, and HPMC can influence the mechanical properties of HPMC/PVA/Xanthan gum films and should be taken into account in the development of the film with desirable properties. Films with a higher concentration of HPMC are tougher, while films with a higher amount of PVA are softer. The buffering capacity of the film F48 was 0.47 mEq compared with 0.33 mEq for the gel composition. The buffering capacity of Film F68 was 0.40 mEq compared with 0.33 mEq for the gel. The TS of film F68 was 1.56, which is close to VCF 1.747. The effect of varying the concentration and use of different grades of HPMC, PVA, and glycerol on the TS was evaluated. Tough film with higher TS and low elongation is possible by using a higher ratio of HPMC. Softer film with moderate TS and high elongation is possible with higher Mw PVA.

### Example 4: Improvement of the Mechanical Properties of Film of PVA, HPMC, and Xanthan Gum

Next, experiments were done to improve the mechanical properties of film F48 by manipulating the proportion of the polymers and glycerol (add additional higher Mw PVA to the polymer blend). Due to the high HPMC concentration used in the film fabrication, Films 68 and 69 are characterized by low elongation (6.72 and 7.37), poor toughness (0.02 and 0.03), and opaque cosmetic appearance. To overcome this limitation, there was a need to fabricate film with moderate TS and high elongation. This aspect was to fabricate potential films with moderate TS and high elongation.

The approach for improving the TS involved manipulating the proportion of the polymers and plasticizer. By varying polymer and plasticizer proportion/concentration, films with different TS and elongation can be fabricated. It was obvious from previous experiments that to achieve films with desired mechanical characteristics similar to VCF, the addition of more polymers and optimization of glycerol ratio was necessary. The higher concentration of HPMC affected the cosmetic appearance of the film significantly. Machado et al reported that women value the aesthetic properties of the vaginal film when considering usage and acceptance (Machado et al. J Pharm Sci 102 (7): 2069-2081 (2013)). For this reason, only PVA and glycerin were considered for another round of film optimization. Two additional vaginal film formulations were prepared by varying concentrations of PVA and plasticizer F70 and F71. The impact of the concentration of the polymer and glycerol on TS, elongation, toughness, and dispersion was evaluated. The films were further considered for acid buffering capacity.

Films comprising PVA, HPMC, Xanthan gum as film-forming polymers, and glycerol as a plasticizer were prepared by solvent-casting. Table 9 presents the weight of the different polymers and excipients used in film fabrication. The film was then collected and cut into smaller fractions of appropriate dimensions. The optimized film was compared with VCF. The films were also evaluated for various parameters such as aesthetic properties (appearance, color, flexibility, and peelability. For the preliminary dispersion test, film sections (5×5 cm) were immersed in preheated 10 ml normal saline at 37° C. and 20 rpm orbital shaking. The time required for complete dispersion was determined visually.

TABLE 10
Tensile strength results for F70 and F71

| | Sample 1 | Sample 2 | Sample 3 | Average | |
|---|---|---|---|---|---|
| Film | (Mpa) | (Mpa) | (Mpa) | reading (Mpa) | SD |
| F48 | 1.25 | 1.12 | 1.11 | 1.16 | 0.07 |
| F70 | 0.92 | 0.90 | 0.88 | 0.90 | 0.02 |
| F71 | 1.23 | 1.21 | 1.28 | 1.24 | 0.03 |

TABLE 11
% elongation results of F70 and F71

| | % Elongation | % Elongation | % Elongation | | |
|---|---|---|---|---|---|
| Films | Sample 1 | Sample 2 | Sample 3 | Average | SD |
| F48 | 24.4 | 30.3 | 29 | 27.91 | 3.81 |
| F70 | 37.9 | 33.47 | 31.75 | 34.37 | 3.17 |
| F71 | 35.4 | 29.76 | 31.51 | 32.22 | 2.89 |

TABLE 12
Toughness results for F70 and F71

| | Toughness | Toughness | Toughness | | |
|---|---|---|---|---|---|
| | (MJ/m3) | (MJ/m3) | (MJ/m3) | Average | |
| Films | Sample 1 | Sample 2 | Sample 3 | (MJ/m3) | SD |
| F48 | 0.16 | 0.25 | 0.2 | 0.2 | 0.04 |
| F70 | 0.25 | 0.22 | 0.28 | 0.25 | 0.03 |
| F71 | 0.27 | 0.23 | 0.21 | 0.23 | 0.03 |

TABLE 13
Results from various tests for F48, F70, and F71

| | Buffering | Dispersion | Mucoadhesive | pH after | Viscosity in | Viscosity in |
|---|---|---|---|---|---|---|
| | Capacity | Time | Strength | dispersion | saline | SVF |
| F48 | 0.47 | 2.48 +/− 0.11 | | | | |
| F70 | 0.5 | 2.38 +/− 0.13 | 0.72 ± 0.067N | | 4 | 23 |
| F71 | 0.44 | 4.02 +/− 0.3 | | | | |
| Phexxi | 0.33 | | 0.2-0.3N | | 940 | 1070 |

| | | Tensile | % | | |
|---|---|---|---|---|---|
| | Stability | Strength | elongation | Toughness | |
| F48 | | 1.16 +/− 0.08 | 27.91 +/− 3.82 | 0.159 +/− 0.05 | Film F29-F48 were prepared by |
| | | | | | the solvent casting method. |
| F70 | | 0.90 +/− 0.02 | 34.37 +/− 3.17 | 0.25 +/− 0.03 | |
| F71 | | 1.24 +/− 0.03 | 32.22 +/− 2.89 | 0.23 +/− 0.03 | |
| Phexxi | | | | | |

TABLE 14
Mechanical properties of VCF

| | Tensile strength (Mpa) | % Elongation | Toughness |
|---|---|---|---|
| Average | 1.747 ± 0.14 | 25.73 ± 4.67 | 0.326 ± 0.09 |

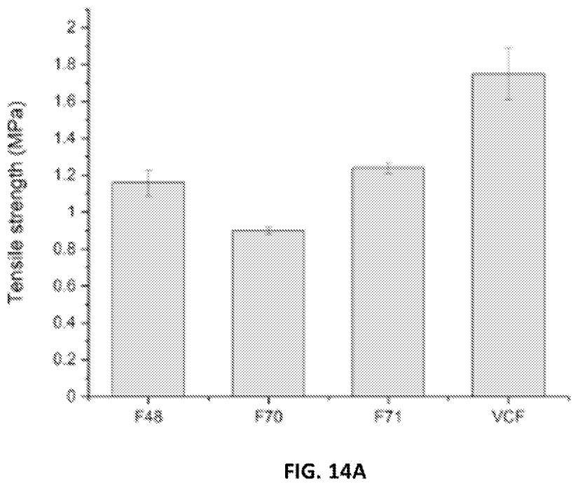

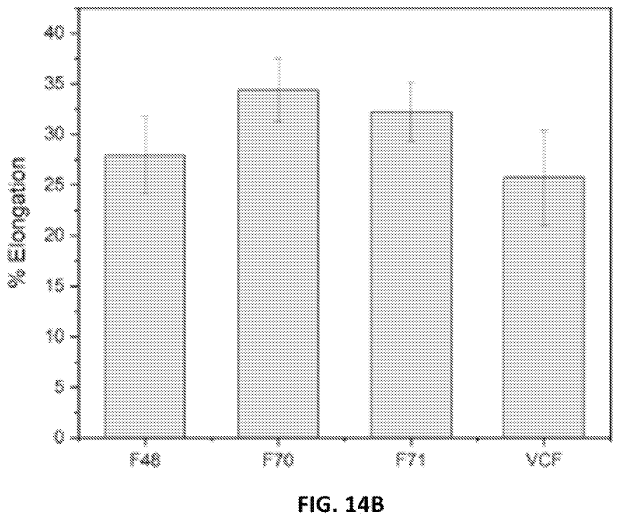

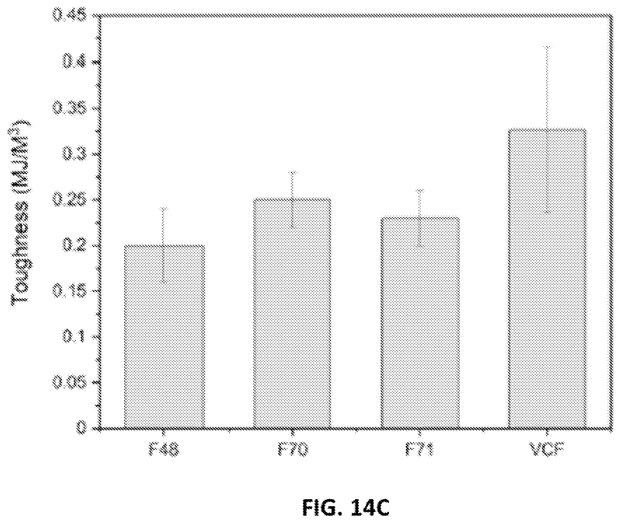

All films were flexible and easy to peel from the casting Petri dish, with a feeling that was smooth to the touch. Unlike film 68 which was hard and less flexible due to the high proportion of HPMC, films F70 and 71 displayed moderate TS and elongation (FIGS. 14A and 14B, Table 15). Toughness is shown in FIG. 14C and Table 15. In this study, VCF was used as a standard. The use of the high molecular weight of PVA leads to a slight increase in TS as seen in film 71. The small increase in PVA used for film F70 did not result in any remarkable increase in TS because of the simultaneous increase in glycerol use. An increase in glycerol results in a flexible film with higher elongation. All the films had good acid buffering capacity, better than the gel. With a target of disintegration time less than 2-3 minutes, film F48 and F70 dissolved rapidly within 3 minutes after exposure to normal saline. Film 71 dissolved within 4 minutes, possibly due to the use of higher molecular weight PVA in the film composition.

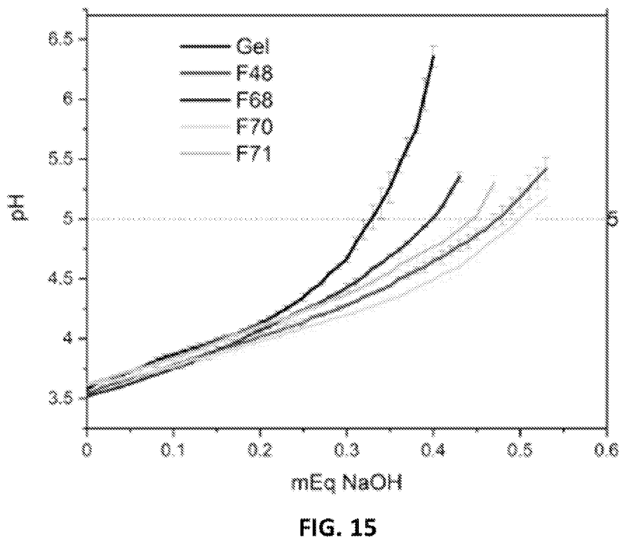

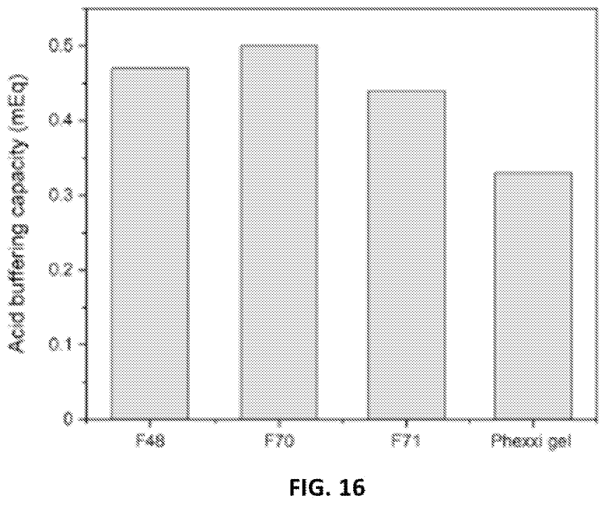

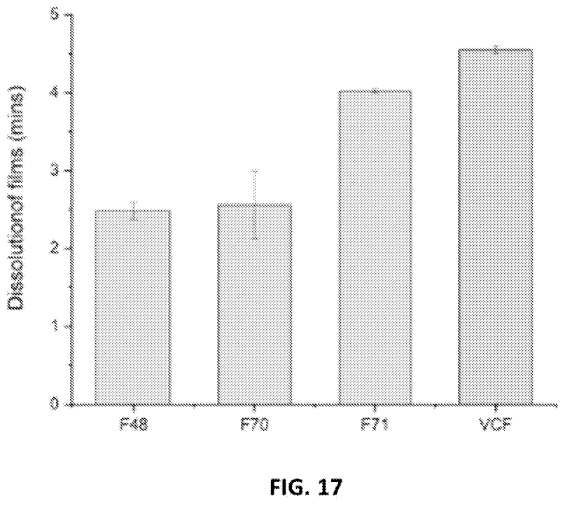

In conclusion, films with more glycerol were more flexible and showed higher elongation at break. The tensile strengths of film 48, 70, and 71 were lower than that of VCF. The % elongation of films F48, 70, and 71 were comparable/slightly higher than that of VCF. Film 71 prepared by adding additional high molecular weight PVA demonstrated a slight improvement in mechanical properties. Film F48 and F70 dissolved rapidly within 3 mins, while film 71 dissolved within 4 mins (FIG. 17). Film dispersion was slightly better than VCF. The acid buffering capacity of films 48, 70, and 71 was higher than that of the gel composition (FIGS. 15 and 16).

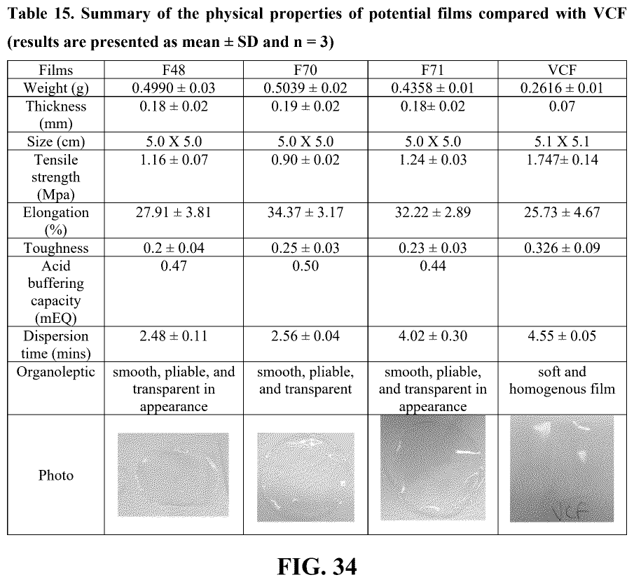

Table 15. Summary of the physical properties of potential films compared with VCF (results are presented as mean±SD and n=3) (See FIG. 34)

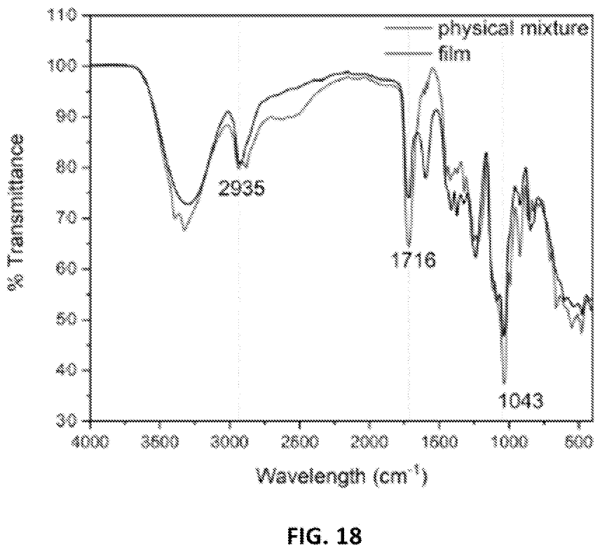

The FTIR spectra of film F70 show that some of the bands have shifted and intensities of some of the bands are decreased. The possible explanation for this is that there is a formation of new material-film. FIG. 18 shows a slight difference in the FTIR spectrum of the cast film compared to the physical mixture which suggests that all the acid ingredients and polymers are well dispersed in the film after solvent evaporation (Lin et al. Thermochimica Acta 254:153-166 (1995)).

### Example 5: Improvement in Mechanical Strength of Investigative Film F70 and F71 by Adjusting PVA

The next set of experiments aimed to improve the mechanical properties of the investigative films F70 and F71 by manipulating only the proportion of the polymers while keeping the glycerol ratio constant (add higher Mw PVA to the polymer blend). Films F72 and F73 were prepared using the solvent casting method. Tables 16 and 17 show the weight of the different polymers PVA, HPMC, Xanthan gum, glycerol as a plasticizer, and acid excipients used in the film fabrication. The mechanical properties of the films were determined using a Texture Analyzer (TA.XT.plus Texture Analyzer, Stable Micro Systems, U.K.) as previously described by Garg et al. (Pharm Res 22 (4): 584-595 (2005)), but slight changes to the size of the film tested. From the instrument software, the tensile strength and elongation at break were determined.

TABLE 16
Composition of film F72 and F73

| | PVA | PVA | | | | | | |
|---|---|---|---|---|---|---|---|---|
| | 30,0000- | 85,000- | PVA | | | | | |
| | 70,000 | 124,000 | 85,000- | | | | Acid | |
| Film | (HD: 86-89) | (HD: 86-89) | 124,000 | HPMC | Xanthan | Glycerol | buffering | Water |
| sample | mg | mg | (HD: 86-89) | E50 mg | gum mg | (mL) | agents | (QS) |
| F72 | 3004.07 | 600 | | 250 | 40 | 0.85 | ✓ | 50 |
| F73 | 3005.9 | | 400 | 250 | 40 | 0.925 | ✓ | 50 |

TABLE 17
The composition of the acid buffering ingredient

| | New composition |
|---|---|
| Ingredients | Amount/Weight (mg) |
| Citric acid (mg) | 200 |
| Lactic acid (μL) | 675 |
| Tartaric acid (mg) | 125 |
| Benzoic acid (mg) | 12.5 |
| Sodium hydroxide (mg) | 175 |

TABLE 18
Composition of F48, F70, and F71 fabricated films

| | | |
|---|---|---|
| | | % (w/w) |
| | | of film |
| 50 ml | 100 ml | ingredients* |

Film F48

| | | | |
|---|---|---|---|
| PVA 30,000-70,000 (mg) | 3000 | 6000 | 22.01 |
| HPMC K15 (mg) | 250 | 500 | 1.834 |
| Xanthan gum (mg) | 40 | 80 | 0.29 |
| Glycerol (ml) | 0.90 | 1.80 (2.33 g) | 8.324 |
| Citric acid (mg) | 2000 | 4000 | 14.67 |
| Tartaric acid (mg) | 1250 | 2500 | 9.17 |
| Benzoic acid (mg) | 125 | 250 | 0.92 |
| NaOH (mg) | 1750 | 3500 | 12.84 |
| Lactic acid (ml) | 6.750 | 13.5 (8.16 g) | 29.94 |
| Water (QS) ml | 50 | 100 | |

Film 70

| | | | |
|---|---|---|---|
| PVA 30,000-70,000 (mg) | 3000 | 6000 | 21.34 |
| PVA 85,000-124,000 (mg) | 400 | 800 | 2.84 |
| HPMC E50 (mg) | 250 | 500 | 1.78 |
| Xanthan gum (mg) | 40 | 80 | 0.28 |
| Glycerol (ml) | 0.925 | 1.85 (2.33 g) | 8.29 |
| Citric acid (mg) | 2000 | 4000 | 14.23 |
| Tartaric acid (mg) | 1250 | 2500 | 8.89 |
| Benzoic acid (mg) | 125 | 250 | 0.89 |
| NaOH (mg) | 1750 | 3500 | 12.45 |
| Lactic acid (ml) | 6.750 | 13.5 (8.16 g) | 29.02 |
| Water (QS) ml | 50 | 100 | |

Film 71

| PVA 30,000-70,000 (mg) | 3000 | 6000 | 21.44 |
|---|---|---|---|
| PVA 146,000-186,000 (mg) | 300 | 600 | 2.14 |
| HPMC E50 (mg) | 250 | 500 | 1.79 |
| Xanthan gum (mg) | 40 | 80 | 0.286 |
| Glycerol (ml) | 0.95 | 1.90 (2.395 g) | 8.56 |
| Citric acid (mg) | 2000 | 4000 | 14.29 |
| Tartaric acid (mg) | 1250 | 2500 | 8.93 |
| Benzoic acid (mg) | 125 | 250 | 0.89 |
| NaOH (mg) | 1750 | 3500 | 12.51 |
| Lactic acid (ml) | 6.750 | 13.5 (8.16 g) | 29.16 |
| Water (QS) ml | 50 | 100 | |

*assumes 0% water remaining in thin film after drying

TABLE 19
Tensile strength result
F73 - Thickness of 0.18 ± 0.02

| | Sample 1 | Sample 2 | | | |
|---|---|---|---|---|---|
| Film | (Mpa) | (Mpa) | Sample 3 | Average | SD |
| F72 | To repeat | To repeat | To repeat | | |
| F73 | 1.412 | 1.417 | 1.53 | 1.45 | 0.067 |

TABLE 20
Elongation results for F72 and F73

| | % | % | % | Average | |
|---|---|---|---|---|---|
| | Elongation | Elongation | Elongation | reading | |
| Films | Sample 1 | Sample 2 | Sample 3 | (Mpa) | SD |
| F72 | To repeat | To repeat | To repeat | To repeat | |
| F73 | 11.94 | 11.05 | 13.5 | 12.16 | 1.24 |

TABLE 21
Toughness results for F72 and F73

| | Toughness | Toughness | Toughness | Average | |
|---|---|---|---|---|---|
| Films | (MJ/m3) | (MJ/m3) | (MJ/m3) | (MJ/m3) | SD |
| F72 | To repeat | To repeat | To repeat | To repeat | |
| F73 | 0.258 | 0.27 | 0.245 | 0.26 | 0.013 |

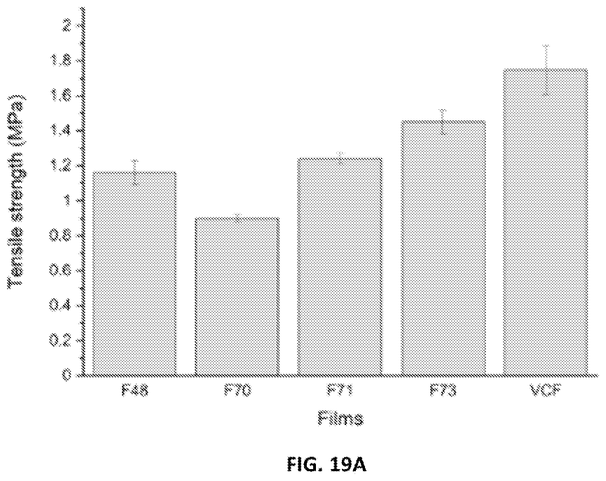

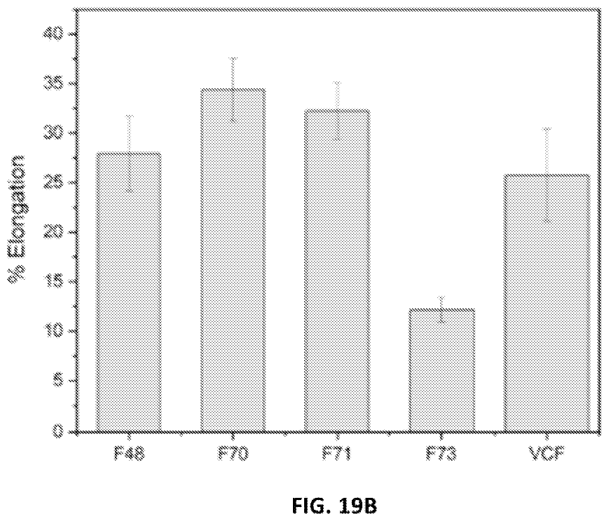

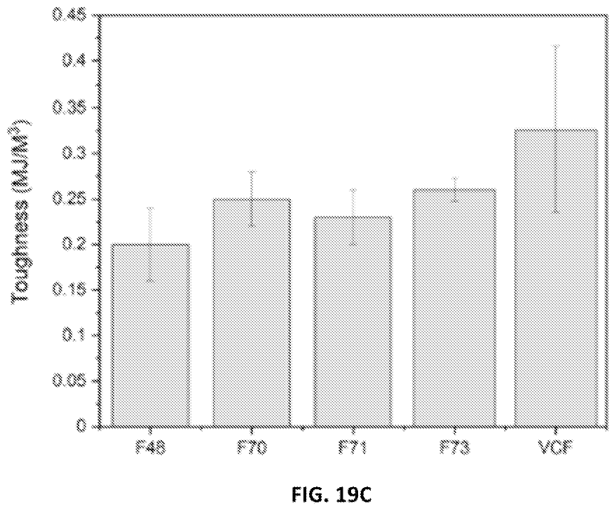

Film 72 was not characterized due to the presence of air bubbles in the film. From the result of the mechanical test, the TS of film 73 improved with an increase in PVA Polymer Mw 85,000-124,000. However, keeping the glycerol ratio constant results in films with poor elongation and flexibility. The TS increase can be attributed to an increase in PVA. The film was slightly rigid with poor elongation (FIGS. 19A-19C). These films are not soft. There is a need to manipulate both PVA and Glycerol for a better outcome.

New composition film (size 5×5 cm and thickness 0.19±0.01) was dispersed in 2 ml SVF until a uniform dispersion was formed, while maintaining the temperature at 37° C. The pH of the mixture before and after the dispersion was then measured using a calibrated pH meter. The data are shown in Table 22.

TABLE 22

| | pH of SVF before dispersing | pH of SVF after dispersing |
|---|---|---|
| Film 70 | film | film |
| Sample 1 | 4.24 | 3.68 |
| Sample 2 | 4.24 | 3.62 |
| Sample 3 | 4.24 | 3.61 |
| Average | 4.24 | 3.64 |

From the experiment, film 70 shows an acid buffering effect towards 2 ml SVF by lowering the pH from 4.24 to 3.64. These results support the assumption that when the films are subjected to the diluting effect of the vaginal fluid (SVF), the film will still maintain the acid buffering ability. This result obtained was similar to the value obtained for the gel composition.

### Example 6: Investigation of the Thermal Stability of New Composition Film by Using FTIR

In the present study, a highly specialized and powerful Fourier transform infrared (FTIR) spectroscopy was used to examine the thermal stability of vaginal film F70. FTIR spectroscopy was carried out on the fabricated film to confirm the stability of the polymers and excipients after subjecting the film to high temperature at 50° C. for 1 week in the oven.

The compatibility of the polymers used in the fabrication of the film (PVA, HPMC, and Xanthan gum), plasticizer, and the acid buffering excipients (citric acid, tartaric acid, benzoic acid, and lactic acid) was studied using the ATR-FTIR instrument Perkin Elmer spectrum 400 USA. The fabricated film sealed in an aluminum foil package was subjected to a high temperature at 50° C. for 1 week. The spectra before and after the test were obtained at a frequency range of 4000-450 cm⁻¹ with a resolution of 4 cm⁻¹ and 16 scanning rates.

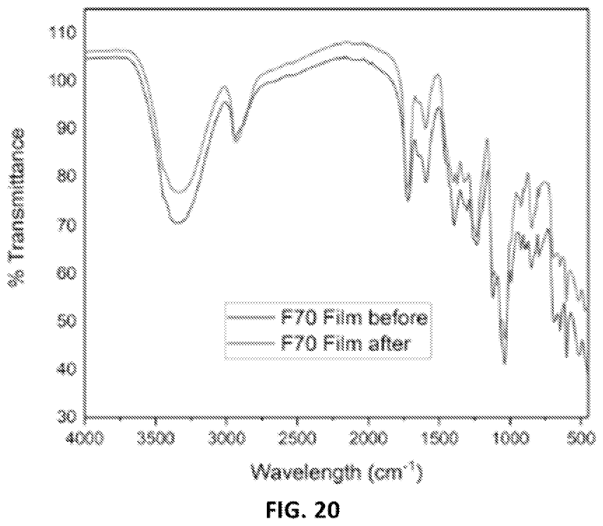

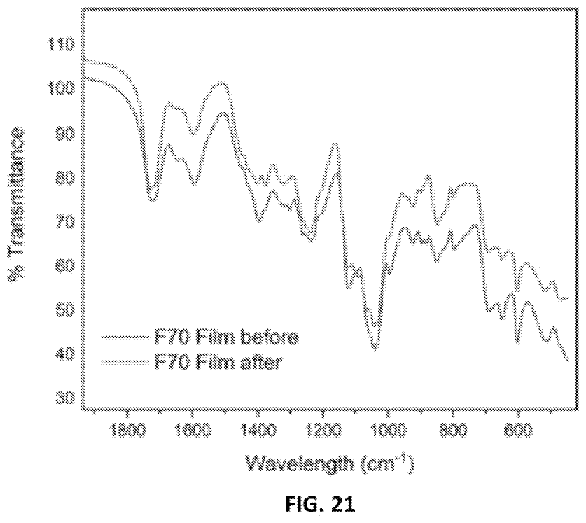

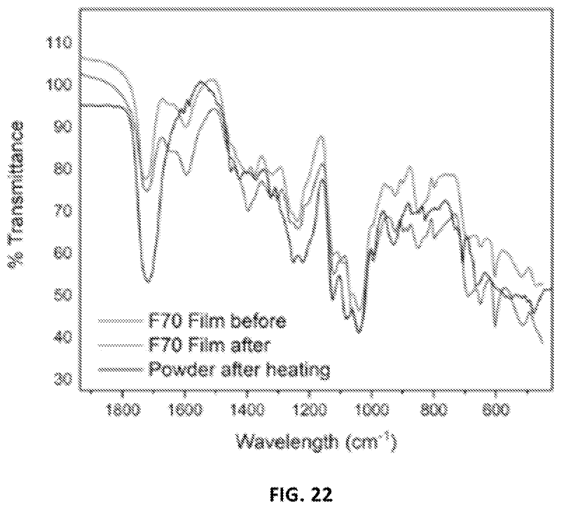

Spectra of the prepared film before and after 1 week in the oven were compared for any possible structural change that may be induced by subjecting the sample to high temperature (FIG. 20). The FTIR results confirm that there is no chemical interaction between the polymers, excipients, and acid buffering agents. Visual examination of the spectra of the freshly prepared film and after 1 week in the oven (50° C. for 1 week) reveals that there is no appreciable difference in spectra apart from a decrease in band intensity and a slight shift in peak position (FIGS. 20 and 21). The reduced peak intensity in the hydroxyl group region (3500-3300 cm⁻¹) can be attributed to dehydration of the film samples due to the high temperature (Doblies et al. Polymers 11 (2): 363 (2019)). Therefore, it is reasonable to say that all the acid buffering ingredients, polymers, and plasticizer used in the fabrication of the new composition film are stable. However, as expected there was a change in the texture of the film due to dehydration. The film became slightly hard, less flexible with a rough surface. It is possible that by removing all the residual water from the film, there may be crystallization of some of the ingredients. From this preliminary result, the film was no longer soft and flexible.

The FTIR results show that no significant alteration occurs in the spectra of the film after subjecting it to heat at 50° C. for 1 week. The result from the FTIR will need to be complemented with the DSC result to make a meaningful conclusion about the thermal stability of the film.

### Example 7: Mucoadhesion Assessment of the New Composition Film, Gel Composition, Film F70, and VCF

Mucoadhesion investigation is very important for the successful formulation of a vaginal film because this aspect determines if the formulation is capable of maintaining a higher amount of API at the site of administration as well as prevention of quick expulsion. Mucoadhesion can help predict how fast the formulation will last before been expelled from the vagina. To investigate the mucoadhesion capacity of the fabricated film, the force required to detach the film was determined by using a TA.XTPlus Texture Analyser (Stable Micro Systems) based on a method previously reported by Professor Garg. The TA.XTPlus texture analyzer (Stable Micro Systems TA-XT. plus Texture Analyzer Godalming, UK) with a mucoadhesion rig was employed. The cellophane membrane (Thermo Fisher Scientific) was a substitute to replace the vaginal tissue in this study.

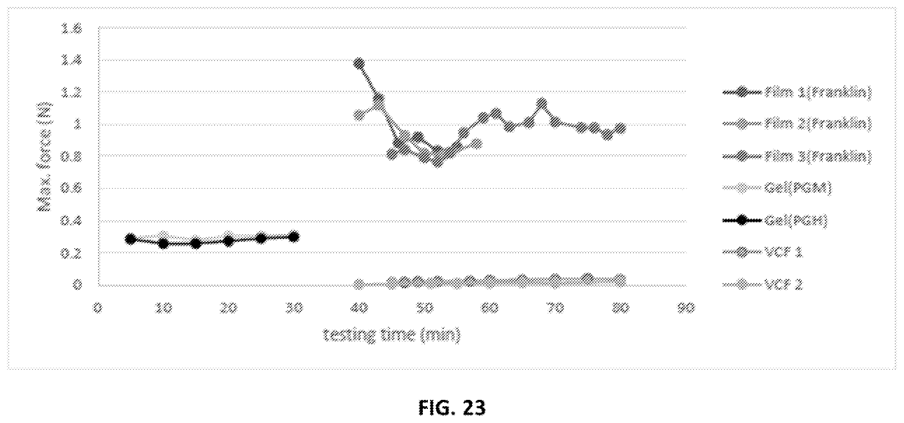

About 1 g gel composition was loaded into the rig, and 5 min after adding the gel, the test was started. One pack (about 0.5-0.6 g/per film) of the film prepared by Franklin was folded and put into a small weight tray (5 ml, 35× 35 mm); 1.5 ml SVF was added; after 25 min at room temperature, the lumps in the gel were gently stirred using a lab stainless steel spatula for 10 min, and then the gel was fully transferred into the rig. The gel stayed in the rig at 37 degrees for 5 min before the tests were performed. One pack (about 0.23 g/per film) of VCF was folded and put into a small weight tray (5 ml, 35×35 mm); 1.5 ml SVF was added after 25 min at room temperature, some small lumps in the gel were gently stirred using a lab stainless steel spatula for 10 min, and then the gel was fully transferred into the rig. The gel stayed in the rig at 37 degrees for 5 min before the tests were performed. (The test time was selected according to the instruction of how to use VCF, “insert VCF, not less than 15 min, and not more than 3 hours intercourse”.) Several measurements were performed for each sample. The results are shown in FIG. 23.

1.5 ml is the minimum amount of SVF required for one film (Franklin prepared). Mucoadhesion of one pack of VCF in 1.5 ml SVF was quite low. The film Franklin prepared has much higher mucoadhesion and that is higher than gel composition without any dilution with SVF (FIG. 23). The mucoadhesive capacity and residence time of the film F70 were higher. Based on the mucoadhesion results, the possibility of the film been expelled from the vagina is unlikely. The strong mucoadhesive properties will confer prolonged effectiveness since the film will be retained in the vagina for a longer period. The strong mucoadhesive layer around the vagina can also help act as a barrier against STI pathogens. The mucoadhesive ability of film F70 is about 3 times higher than the gel composition, which may likely translate to a longer pre-coital lead-time than the current one hour for the gel. The strong mucoadhesive property of Film F70 will likely increase the residence time of the film in the vagina.

From the preliminary result obtained from FIG. 23, it is evident that film F70 possesses strong mucoadhesive property and it will be attached to the vaginal mucosa at the time of administration. The result obtained shows that F70 displays better mucoadhesion capacity than the gel composition and VCF. The strong mucoadhesive layer can act as a barrier against STI Pathogens.

### Example 8: Stability of Film F70

Stability evaluation of the new composition film will help support the proposed shelf-life of the film. It is expected that the physiochemical properties of new composition film such as acid buffering, mechanical properties, dispersion time was maintained as well as remained unchanged in the film formulation throughout the stability assessment period.

Films were individually sealed in aluminum foil packages and stored at 25° C./65% RH for 12 months as a long-term study and 40° C./75% RH for 6 months as an accelerated study. At a specific time point, film samples were pulled for physicochemical characterization. Testing of the films was conducted at 0, 0.5, 1, 2, 3, and 6 months. At each time the following characterization was evaluated:
Appearance Acid buffering capacity
Film dispersion time in SVF at 37° C.
Moisture content and absorption based on the change in weight
Viscosity of films after dispersion
Mechanical strength (TS and elongation).

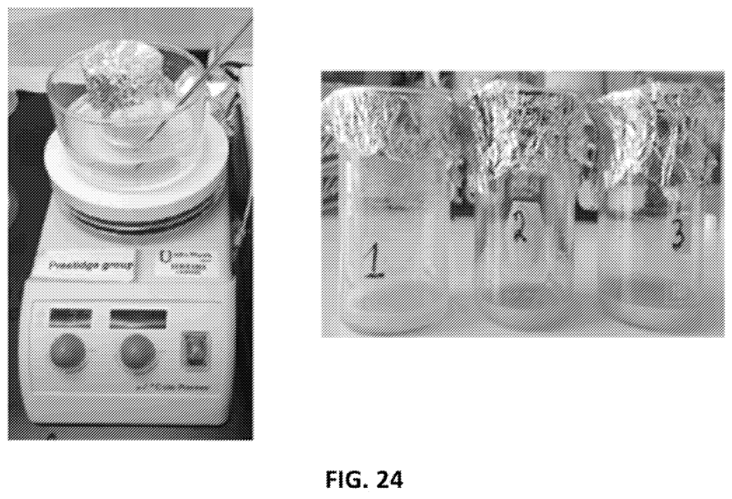

The behavior of films in SVF at 37 degrees was monitored visually and the time required for the film to form gel was recorded (FIG. 24). The resulting dispersion was further used to determine the pH using a PH Meter. The mechanical properties of the films were determined using a Texture Analyzer (TA.XT.plus Texture Analyzer, Stable Micro Systems, U.K.) as previously described by Garg et al. (Pharm Res 22 (4): 584-595 (2005)), but slight changes to the size of the film tested. From the instrument software, the tensile strength and elongation at break were determined. For the preliminary dispersion test, film sections (5×5 cm) were immersed in preheated 5 ml SVF at 37° C. The time required for complete dispersion of the film into gel was determined visually.

### Example 9: Determination of the Buffering Capacity of Film F70

This aspect involves investigating the acid buffering capacity of the film F70 for stability purposes at a time point (zero). The amount of sodium hydroxide required to raise the pH of 5.0×5.0 cm of the film from pH 3.5 to 5 was determined using titration.

TABLE 23
Result of the dispersion and pH of Film 70

| | pH of SVF | pH of SVF | Time required for |
|---|---|---|---|
| | before | after | the film to form a |
| | dispersing | dispersing | gel in 5 ml |
| Film 70 | film | film | SFV (minutes) |
| Sample Film 70-1 | 4.21 | 3.68 | 2.39 |
| Sample Film 70-2 | 4.21 | 3.67 | 2.24 |
| Sample Film 70-3 | 4.21 | 3.66 | 2.50 |
| Average | 4.21 | 3.67 ± 0.01 | 2.38 ± 0.13 |

F70 dispersed rapidly within 2.38 mins after exposure to 5 ml SVF. The film also shows an acid buffering effect towards 5 ml SVF by lowering the pH from 4.21 to 3.67 (Table 23). These results helped with the prediction and assumption that when the new composition films are diluted with SVF, the film will still maintain the acid buffering ability.

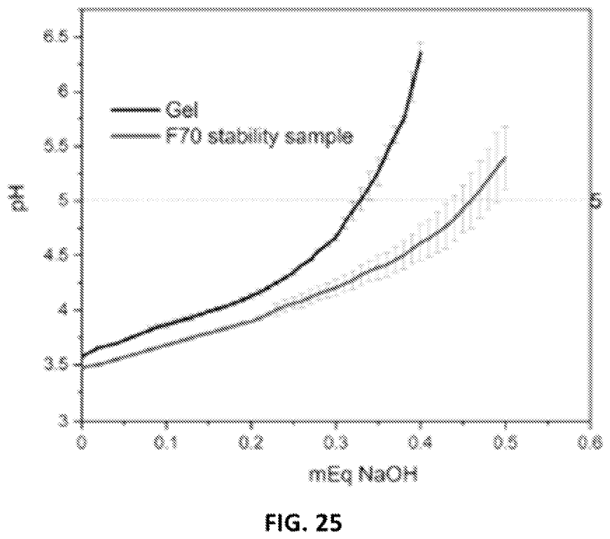

Further characterization of film F70 for stability purpose shows that the acid buffering capacity was 0.46 mEq compared with 0.33 mEq for the gel composition (FIG. 25).

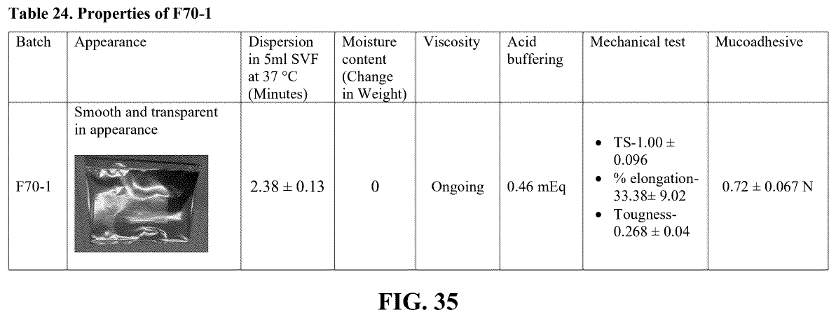

### Table 24. Properties of F70-1 (See FIG. 35)

Preliminary results for the stability of sample F70 (conclusion at time point zero-from ongoing stability) were as follows:
A film with desirable aesthetic properties was fabricated using HPMC, PVA, and xanthan gum
Film F70 was soft, flexible without any evidence of sharp edges
Film mucoadhesive force was 0.72±0.067 N, which is superior to the gel composition
The mechanical results were within an acceptable range
F70 dispersed rapidly within 3 mins (2.38±0.13) after exposure to 5 ml SVF at 37° C.
Film 70 still maintained the acid buffering ability against 5 ml SVF
Acid buffering capacity of film F70 was found to be 0.46 mEq higher than the gel composition.

In conclusion, FTIR results show that significant alterations in the spectra of the film F70 after subjecting it to heat at 50° C. for 1 week. The film became slightly hard, less flexible with a rough surface. Film F70 exhibits higher values of mucoadhesive force when compared to gel composition and VCF. The strong mucoadhesive properties will likely confer on the film a longer pre-coital lead-time. F70 disperses rapidly within 3 mins (2.38±0.13) after exposure to 5 ml SVF at 37° C. Finally, film 70 still maintains the acid buffering ability against SVF.

### Example 10: Investigation of the Thermal Stability of the New Composition Film Using the DSC/XRD Technique

This study aimed to investigate the physicochemical change that can result from heating the film at 50° C. for 1 week in the oven. Any change in the compatibility between the film-forming polymers and the acid buffering agents was investigated. As expected, any kind of incompatibility between the acid buffering ingredients and the film-forming polymer can affect its performance to a significant extent. The effect of heat on the compatibility of the acid buffering ingredients as well as the new composition film was further studied by thermal analysis DSC and XRD.

Differential scanning (DSC) of the physical mixtures (PVA, HPMC, Xanthan gum, citric acid, tartaric acid, benzoic acid), newly fabricated film, and film subjected to heating at 50° C. for 1 week in the oven was investigated using a Discovery DSC TA Instrument (model Discovery DSC 2920, New Castle, DE, USA). Briefly, ~2.0±0.5 mg of each sample was weighed accurately in a Tzero Pans with a lid before being heated from 25 to 250° C. in a nitrogen atmosphere at a rate of 10° C./min. For the XRD analysis, both the newly fabricated film, and film subjected to heating at 50° C. for 1 week in the oven was subjected to X-ray diffraction test using a Malvern Panalytical XRD (Empyrean XRD, UK) with a Cu Kα radiation source (2=1.5406 Å) and with an operating voltage of 40 kV. Both samples were measured from 5 to 50° two theta with a step size of 0.02°.

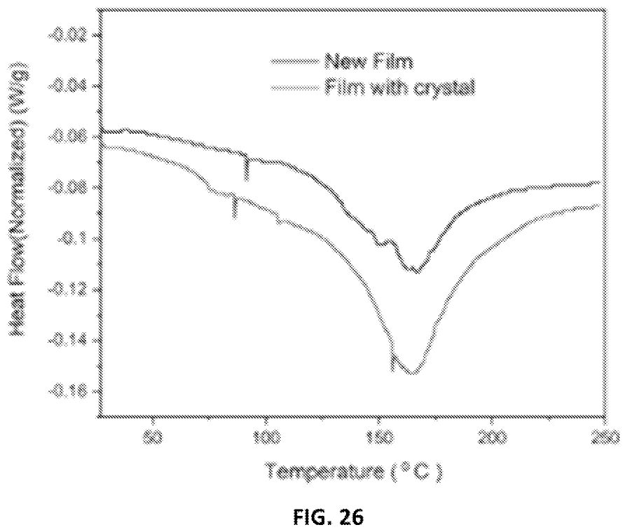

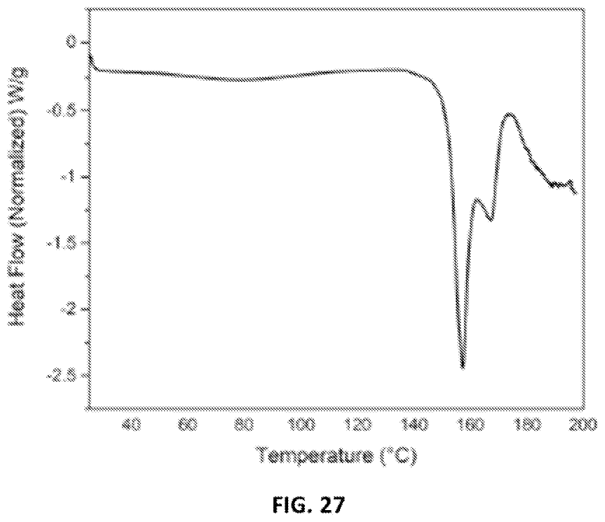

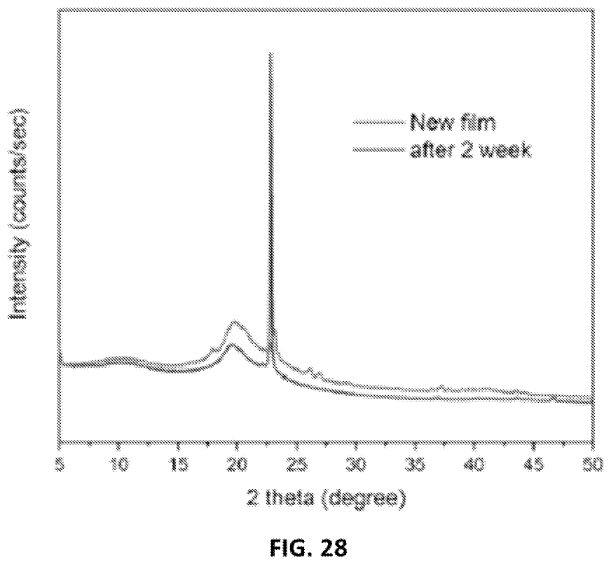

After drying the new composition film at 50° C. for 1 week in the oven, there was evidence of crystals that appeared on the film possibly from the dehydration of residual moisture from the film. This dehydration can likely lead to recrystallization of the API/acid buffering ingredients from the film. The visual examination showed changes in the appearance of the film. From the DSC result, there was a slight change in the DSC of the film with crystal when compared with a new film. There is a small endothermic peak around 156° C., which is absent in the fresh film (FIGS. 26 and 27). The XRD of the fresh film was also slightly different from the film subjected to heating at 50° C. for one week in the oven (FIG. 28). The result of the XRD further complements both the change in visual appearance and the DSC result. The XRD suggests that the film acid buffering agents that were molecularly dispersed in the film-forming polymers are beginning to show crystalline formation with small diffractive peaks from 25-45° C. These low-intensity peaks are more prominent in the film with crystal while these low-intensity peaks are very low in the new film. It is likely that the peak at 157° C. is from citric acid. Citric acid shows a sharp endothermic melting peak at 153° C. In the new film, this peak is absent, suggesting that the acid buffering agent was molecularly dispersed in the film-forming. However, the dehydration and removal of the residual moisture content in the new composition film result in a phase change (crystallinity of the API/acid buffering agents). This ultimately leads to the appearance of crystalline API/drug appearance on the film surface.

The DSC thermogram of the heated film shows a small endotherm peak around 157° C., suggesting that some of the acid buffering agents are beginning to crystallize out of the film due to dehydration from heating the film in the oven. XRD Result shows a slight change in the crystalline component of the sample with crystal when compared with the newly synthesized film.

### Example 11: Determination of the Viscosity of New Composition Film

Vaginal films must possess suitable spreading and retention capability such that they can provide and give adequate protection to the vaginal epithelia from harmful pathogens. The viscosity and dispersibility of vagina formulation in the vaginal environment after administration governs the spreading and retention of the formulations, which is essential to achieve desired efficacy and effectiveness. Considering that vaginal films are likely to be dispersed in the vaginal fluid after application as well as dilution due to semen during sexual intercourse, it is necessary to evaluate the viscosity of the film dispersion. The present study aims to determine the viscosity of the new composition film after it has been dispersed with simulated vaginal fluid SVF (pH 4.2). In this preliminary work, the viscosity of the new composition film was compared with the gel composition. The rheological properties of diluted gel composition batch PGH using SVF (pH 4.2) and new composition film with 5 ml SVF was determined using Rheosys Viscometer at 37° C. over relevant and useful biologically shear rates (0.1-1000). Effective and thorough mixing of vaginal formulation such as gel and films with vaginal fluid will likely result in about 67-80% dilution (Machado et al. J Pharm Sci 102 (7): 2069-2081 (2013)).

The viscosity of the diluted gel was measured using the Rheosys Merlin VR instrument fitted with a 15 mm diameter stainless steel parallel plate assembly and with a gap of 0.5 mm (Table 25). In brief, part of the gel (gel 5 g after dilution with 5 ml SVF-50% dilution) was placed on the bottom plate and the excess gel was removed before starting the test. The experiment was conducted at 37±0.1° C. Three replicates were investigated for batch PGH. The viscosity of the diluted gel in simulated vaginal fluid (SVF) was measured using Rheosys Merlin VR (Scientex Pty Ltd, Melbourne, Victoria, Australia) with a temperature-controlled parallel plate with 15 s of thermal equilibrium. The same procedure was applied for the film (new composition film size-5×5 cm and thickness 0.19±0.02) was dispersed in 5 ml simulated vaginal fluid (SVF) and the viscosity of the resulting dispersion was measured using Rheosys Merlin VR.

TABLE 25
The setting on the Rheosys Merlin VR for the test

| Setting | Parameter/Criteria |
|---|---|
| Measuring plate type | Parallel plate 150 mm |
| Temperature | 25 and 37° C. |
| Start speed | 0.1 |
| End speed | 1000 |
| Number steps | 10 |
| Delay time | 30 sec |
| Integration time | 10 sec |
| Direction | up |

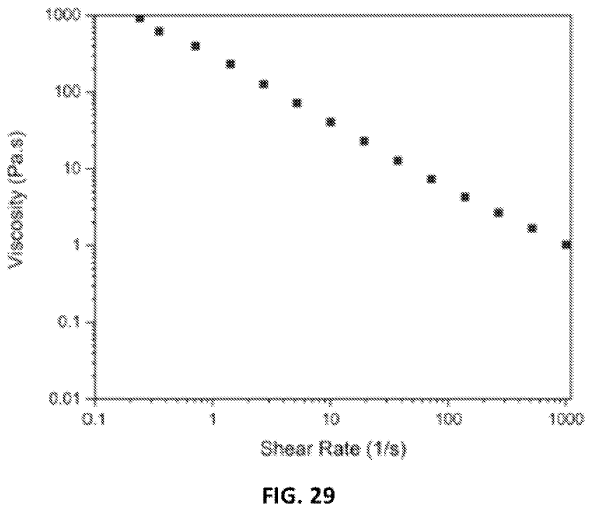

From the result in Table 26 and FIG. 29, the gel composition exhibited shear-thinning behavior. The viscosity of the gel was drastically reduced with an increase in the shear rate from 0.1 to 1000 and the power index of the graph was less than 1, which means that the gel possesses shear-thinning behavior. However, after diluting the film with 5 ml of SVF, the dispersion was very thin, so that it was difficult to obtain any meaningful viscosity reading within a shear rate of 0.1 to 100. The data obtained were inconsistent for the film at a shear rate below 100. For this reason, another method of viscosity measurement was considered.

TABLE 26
Results of three replicate viscosity for diluted sample PGH 50% dilution at 37° C.

| Temperature | Shear Rate | | | | Average | (Pa · s) |
|---|---|---|---|---|---|---|
| (° C.) | (1/s) | Sample 1 | Sample 2 | Sample 3 | (Pa · s) | SD |
| 37.16 | 0.12 | 1580.64 | 1876.77 | 1578.30 | 1678.57 | 171.65 |
| 37.14 | 0.24 | 1024.94 | 899.66 | 853.62 | 926.08 | 88.66 |
| 37.10 | 0.35 | 651.09 | 573.82 | 618.30 | 614.40 | 38.78 |
| 37.06 | 0.71 | 391.16 | 421.93 | 383.71 | 398.93 | 20.26 |
| 37.03 | 1.41 | 226.71 | 243.24 | 223.17 | 231.04 | 10.71 |
| 37.02 | 2.71 | 121.96 | 135.57 | 124.02 | 127.18 | 7.34 |
| 37.02 | 5.18 | 70.60 | 75.96 | 67.88 | 71.48 | 4.11 |
| 37.00 | 10.01 | 38.73 | 44.07 | 38.68 | 40.50 | 3.10 |
| 37.00 | 19.32 | 21.75 | 25.21 | 21.67 | 22.88 | 2.02 |
| 36.99 | 37.35 | 12.15 | 14.18 | 12.06 | 12.80 | 1.20 |
| 37.00 | 71.98 | 6.91 | 8.17 | 6.94 | 7.34 | 0.72 |
| 36.99 | 138.90 | 4.08 | 4.79 | 4.07 | 4.31 | 0.42 |
| 36.99 | 268.25 | 2.56 | 2.94 | 2.51 | 2.67 | 0.23 |
| 37.00 | 517.89 | 1.62 | 1.85 | 1.57 | 1.68 | 0.15 |
| 37.00 | 999.97 | 0.99 | 1.13 | 0.96 | 1.03 | 0.09 |

Gel composition exhibited shear-thinning behavior. From the power-law mathematical model, n is less than 1 (0.83), however this method could not be used for the film because it was not robust enough for the new composition film.

In this case, the viscosity of the film dispersion and gel dispersion was measured at 25° C. using both saline and SVF. In brief, a new composition film (size-5×5 cm) was dissolved in 10 ml SVF and the viscosity of the dispersion was measured using Brookfield Viscometer using spindle S05 at 100 RMP. The stop time for the experiment was 2 minutes for each measurement and triplicated measurement was done. The same procedure was repeated for the gel composition (5 g gel was dispersed with 10 ml SVF). To investigate any change in viscosity due to the dispersion fluid same experiment was repeated using saline in place of SVF.

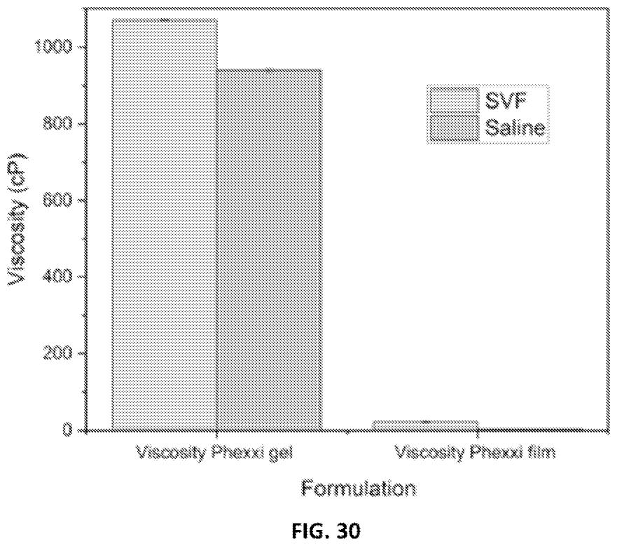

The gel composition was found to possess higher viscosity in comparison to the film (about 46 fold higher; FIG. 30). Also from the result, there is evidence of strong interaction between the gel and the SVF. The viscosity of the gel was higher in SVF compared to saline. For the film, there was a dramatic increase in viscosity from 4 to 23 cP, suggesting that the increase in viscosity coupled with strong bioadhesion of the film will help immobilize both STI pathogens and sperm.

TABLE 27
Viscosity of film and gel using saline and SVF as dilution media

| Formulation type | Viscosity (cP, n = 3) | SD |
|---|---|---|
| Gel with 10 ml SVF | 1070 | 2 |
| Gel with 10 ml saline | 940 | 4 |
| Film with 10 ml SVF at zero time | 23 | 2.65 |
| Film with 10 ml saline at zero time | 4 | 0 |

The gel composition viscosity was 46 fold higher than the new composition film. In this study, the effect of dilution fluid on the viscosity of the vaginal formulation was also investigated. There was an increase in the viscosity of the gel and film upon changing the dilution fluid from saline to SVF.

### Example 12: Evaluation of Stability of the Films

A stability evaluation of the new composition film was performed under the ICH condition. At specific time points—2 weeks and 4 weeks—the new composition film was removed from the packaging material and assay for any change in physical characteristics, dispersion, acid buffering capacity, mucoadhesion, viscosity, and mechanical properties.

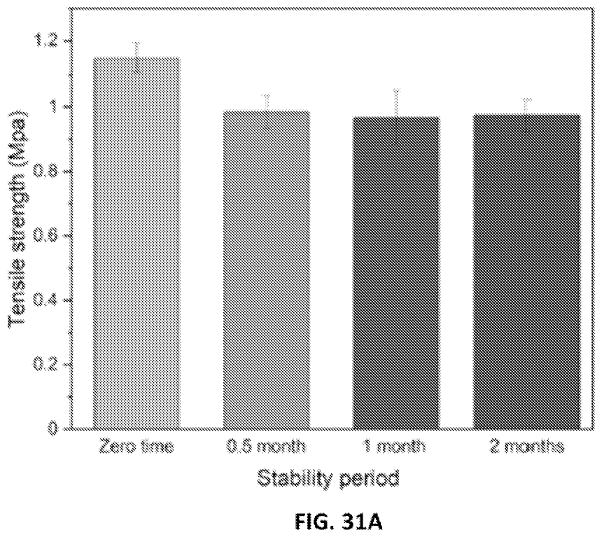

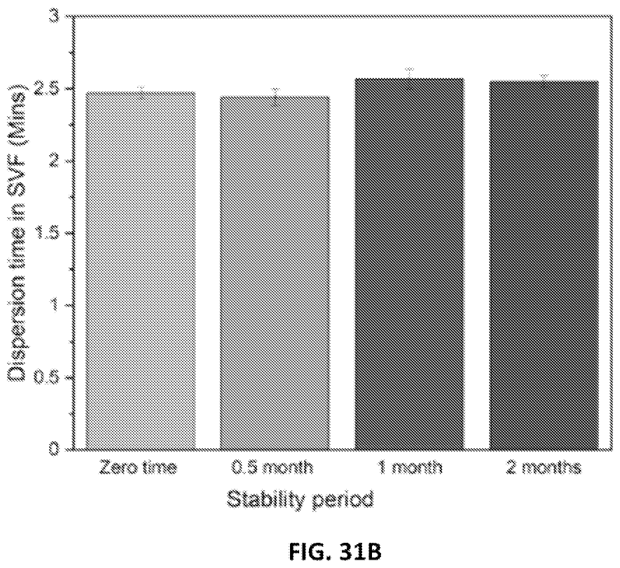

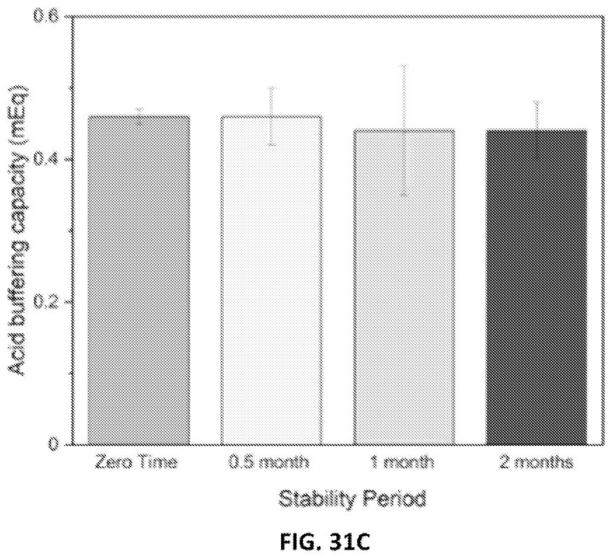

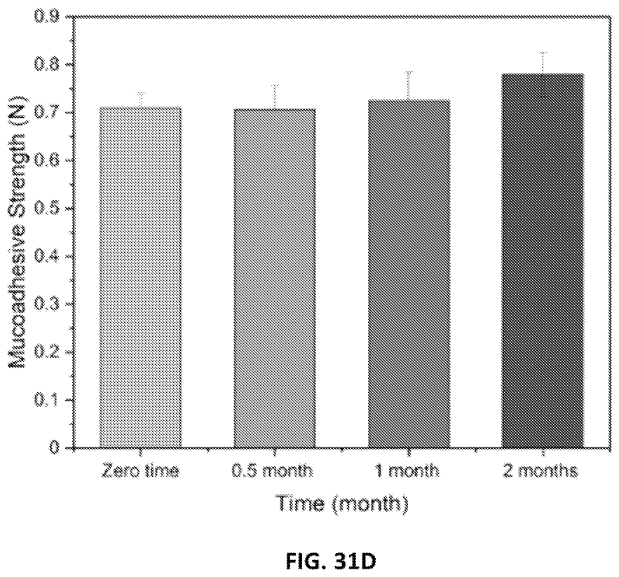

The film samples were evaluated by visual inspection for any change in appearance, mechanical properties using texture analyser, moisture content, dispersion time, and acid buffering capacity at each time point (1 and 2 months). The results are presented in FIGS. 31A-31D. Based on the physicochemical evaluations performed the films were found to maintain stable characteristics at 40° C./75% RH after 1 and 2 months. The buffering capacity and other parameters remained within the acceptable range. There was no increase in moisture content.

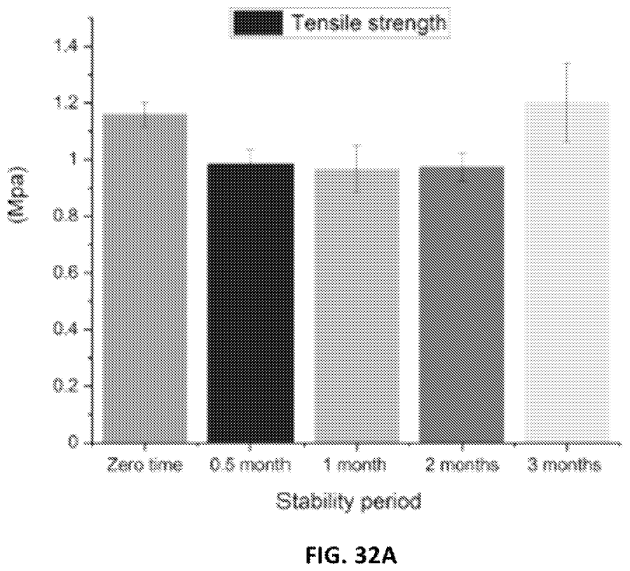

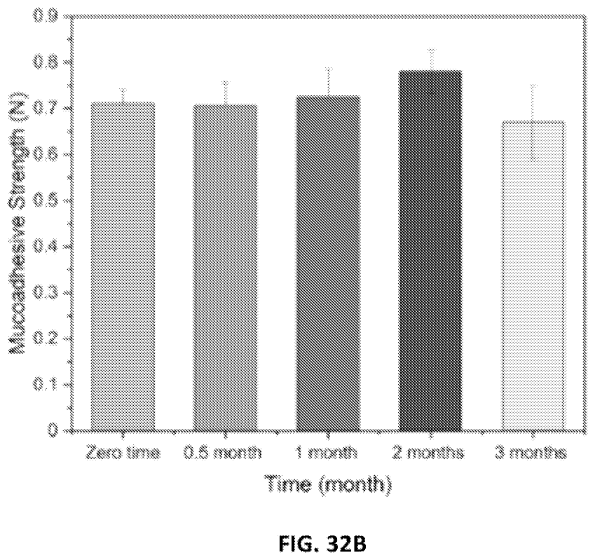

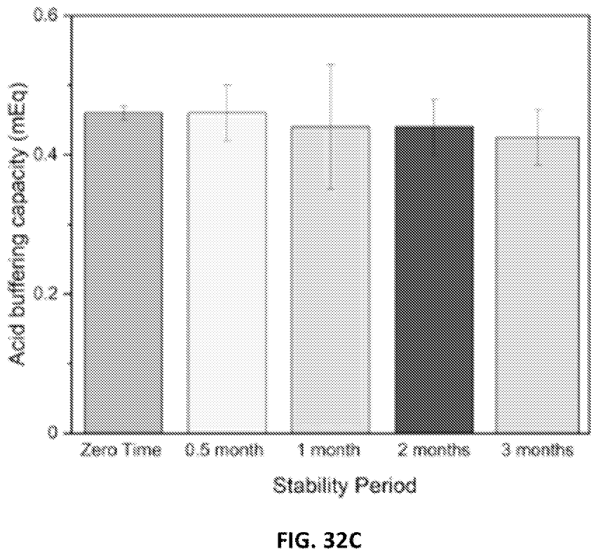

The stability assessments of the new composition films show a slight change in dissolution and mechanical properties. The acid buffering capacity remained stable. The slight change can be attributed to the high temperature, and loss of the residual water content in the film at accelerated stress conditions (40° C./60% R.H.). The films maintained stable characteristics and behavior with less than 10% deviation from the initial starting point. The film's dissolution, acid-buffering capacity, mechanical and mucoadhesive results are presented in FIGS. 32A-32C.

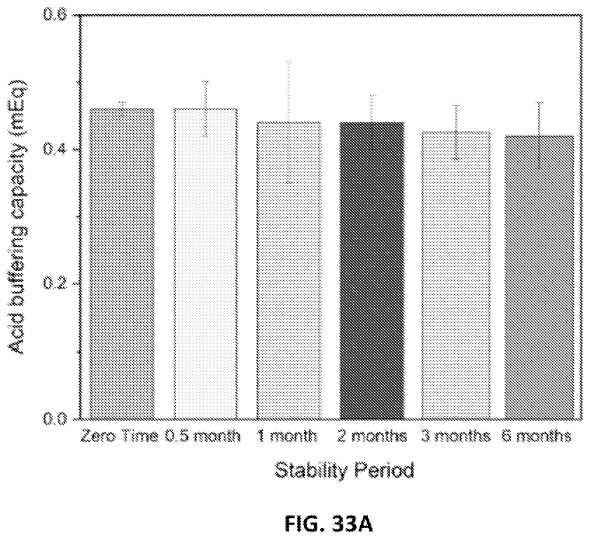

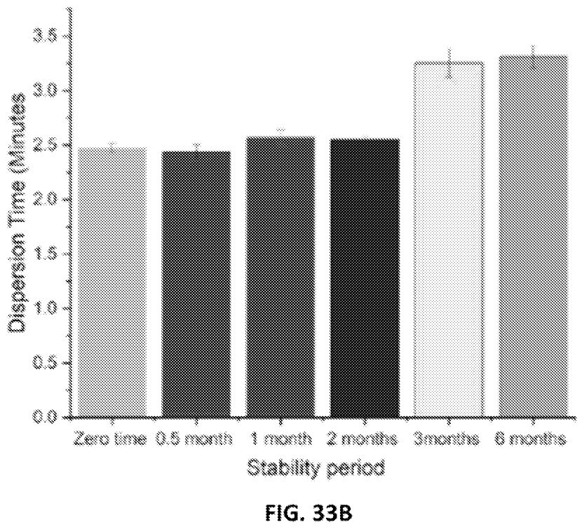

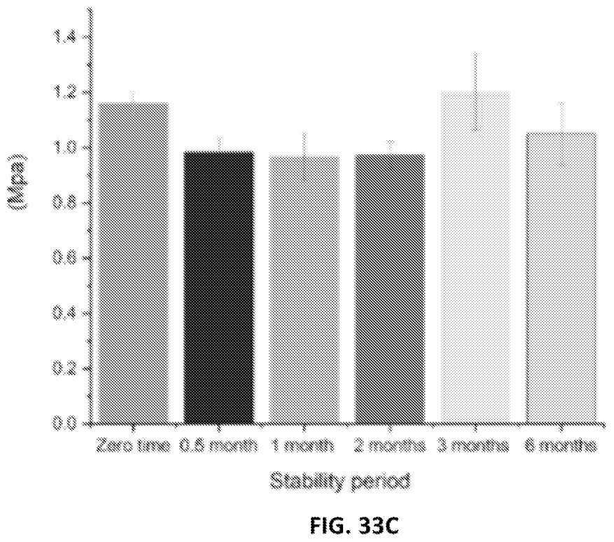

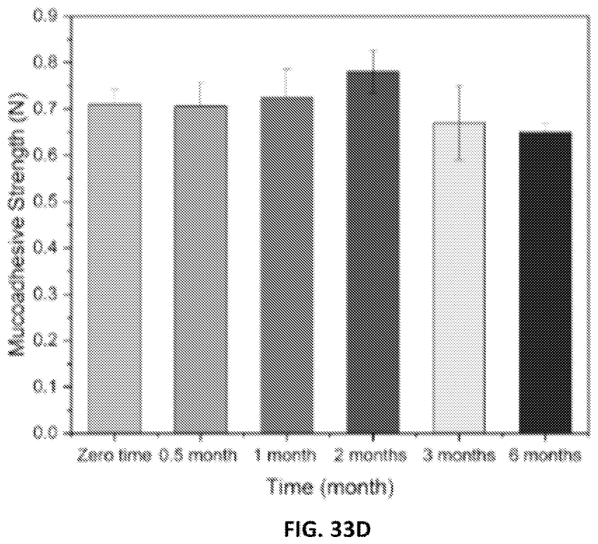

The stability assessments of the new composition films for samples stored at 25° C./60% R.H. did not show any significant change in the visual character of the film. At this condition (25° C./60% R.H.) the films were found to be stable for 6 months. The acid buffering capacity at the end of 6 months was 0.43 mEq compared to the 0.46 mEq at the start of the stability study. A slight deviation in the dissolution, mechanical and mucoadhesive properties compared to the initially manufactured parameter was observed. Overall, there was no significant change at 25° C./60% R.H. (FIGS. 33A-33D). However, there was a slight change in the physical appearance of the film stored at 40° C./75% RH. Previous studies have reported changes in physicochemical properties of films when subjected to accelerated stability testing as per ICH guidelines (J Pharm Innov 12 (2): 142-154 (2017)). At the accelerated stress condition for 180 days, the water content of the film gets reduced which can potentially cause problems such as over-saturation of the acid buffering ingredients in the film. This is the likely reason for the change in the aesthetic appearance of the film. The slight change can be attributed to the high temperature, humidity, and loss of the residual water content in the film. The results are presented in FIGS. 33A-33D. There was a reduction in the acid buffering capacity of the film at both 25° C./60% R.H. and 40° C./75% R.H. However, the deviation at the end of 6 months was less than a 10% deviation from the initial starting point. Overall, the film maintains the desired physicochemical properties within acceptable variation during the stability studies. There was a slight increase in dissolution of the film in SVF, an increase in tensile strength, and a slight reduction in mucoadhesive (FIGS. 33A-33D).

What is claimed is:

1. A thin film comprising:
about 10% (w/w) to about 50% (w/w) L-lactic acid;
about 10% (w/w) to about 30% (w/w) PVA with a molecular weight (MW) between about 30,000 to about 70,000
about 0% (w/w) to about 8% (w/w) PVA with MW bet tween about 85,000 to about 124,000,
about 0% (w/w) to about 6% (w/w) PVA with MW between about 146,000 to about 186,000; and
about 5% (w/w) to about 15% (w/w) glycerol,
wherein the thin film does not comprise alginic acid.

2. The thin film of claim 1, further comprising an additional buffering agent.

3. The thin film of claim 2, wherein the additional buffering agent is an organic acid.

4. The thin film of claim 2, wherein the additional buffering agent is selected from citric acid, tartaric acid, benzoic acid, sodium monocitrate, and any combination thereof.

5. The thin film of claim 1, further comprising a preservative.

6. The thin film of claim 5, wherein the preservative is benzoic acid and/or sodium benzoate.

7. The thin film of claim 1, further comprising hydroxypropyl methylcellulose (HPMC), xanthan gum, carbopol, hydroxyethylcellulose, and any combination thereof.

8. The thin film of claim 7, wherein the thin film comprises about 0.5% (w/w) to about 10% (w/w) HPMC.

9. (canceled)

10. (canceled)

11. The thin film of claim 7, wherein the thin film comprises about 0.05% (w/w) to about 1.5% (w/w) xanthan gum.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The thin film of claim 8, wherein the HPMC is HPMC E50 or HPMC K15.

27. (canceled)

28. (canceled)

29. The thin film of claim 4, comprising about 10% (w/w) to about 20% (w/w) citric acid.

30. The thin film of claim 4, comprising about 4% (w/w) to about 15% (w/w) tartaric acid.

31. The thin film of claim 4, comprising about 0.2% (w/w) to about 2% (w/w) benzoic acid.

32. (canceled)

33. (canceled)

34. The thin film of claim 1, further comprising about 8% (w/w) to about 20% (w/w) sodium hydroxide.

35. (canceled)

36. (canceled)

37. (canceled)

38. (canceled)

39. The thin film of claim 1, wherein the thin film has a bioadhesive strength of about 0.2 to about 1 N.

40. The thin film of claim 1, wherein the thin film has a pH after dispersion in saline of less than about 3.7.

41. The thin film of claim 1, wherein the thin film has a viscosity after dispersion in simulated vaginal fluid (SVF) of at least about 20 cP.

42. (canceled)

43. The thin film of claim 1, wherein the thin film has a tensile strength of between about 0.8 Mpa and about 2 Mpa.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. A method of contraception, comprising intravaginally administering the thin film of claim 1.

98. A method of preventing transmission of a sexually transmitted infection, comprising intravaginally administering the thin film of claim 1.

99-114. (canceled)
